(19) United States
(12) Patent Application Publication
(10) Pub. No.: US 2020/0306343 A1
(43) Pub. Date: Oct. 1, 2020

(54) TREATMENT OF AN ISCHEMIC HEART DISEASE

(71) Applicant: Yeda Research and Development Co. Ltd., Rehovot (IL)

(72) Inventors: Eldad TZAHOR, Rehovot (IL); Kfir Baruch UMANSKY, Rehovot (IL); Rabea HINKEL, Rehovot (IL); Christian KUPATT, Rehovot (IL)

(73) Assignee: Yeda Research and Development Co. Ltd., Rehovot (IL)

(21) Appl. No.: 16/891,165

(22) Filed: Jun. 3, 2020

Related U.S. Application Data
(63) Continuation of application No. PCT/IL2018/051323, filed on Dec. 3, 2018.
(60) Provisional application No. 62/593,942, filed on Dec. 3, 2017.

(51) Int. Cl. A61K 38/17; A61P 9/10

(52) U.S. Cl. CPC A61K 38/1709; A61P 9/10

(57) ABSTRACT

A method of treating an ischemic heart disease in a subject in need thereof is provided. The method comprises administering to the subject a therapeutically effective amount of Agrin in an anterograde intracoronary manner, thereby treating the ischemic heart disease in the subject.

### RELATED APPLICATIONS

This application is a Continuation of PCT Patent Application No. PCT/IL2018/051323 having international filing date of Dec. 3, 2018 which claims the benefit of priority under 35 USC § 119(e) of U.S. Provisional Patent Application No. 62/593,942 filed on Dec. 3, 2017. The contents of the above applications are all incorporated by reference as if fully set forth herein in their entirety.

### SEQUENCE LISTING STATEMENT

The ASCII file, entitled 82821SequenceListing.txt, created on Jun. 3, 2020, comprising 238,048 bytes, submitted concurrently with the filing of this application is incorporated herein by reference.

### FIELD AND BACKGROUND OF THE INVENTION

The present invention, in some embodiments thereof, relates to methods of treating ischemic heart diseases.

In recent years, ischemic heart diseases have become the number one cause of mortality worldwide^{1.2}. One of the most prevalent manifestations of ischemic heart disease is acute myocardial infarction. In this injury, a coronary artery is occluded, in turn causing necrosis, inflammation and scarring of the heart³. The damage caused in this scenario can also lead to ongoing deterioration of the heart, including dilated cardiomyopathy, chronic heart failure (CHF) and even ventricular wall rupture³. Unlike many other tissues, the adult heart in mammals and specifically adult cardiomyocytes (CMs) are mostly post mitotic, and therefore cannot divide to regenerate the damaged tissue. It is therefore essential to investigate ways to promote cardiac regeneration in the affected patients.

Although several regenerative approaches have been suggested and indeed confirmed in small animal models (rodents), as of yet there are no clinically-valid therapeutic options to treat myocardial infarction (MI)⁴. In a broad sense, the current methodologies dichotomizes into two approaches: 1) supplying the heart with exogenous CMs (cardiac stem cells, iPS or trans-differentiation “reprogramming” of cardiac fibroblasts), or 2) reactivation of inherent neonatal regeneration mechanisms, i.e., by employing mitogens or growth factors⁴. As mentioned, both efforts have not yet resulted in a successful clinical therapy. Besides inherent problems with the basic concepts (i. e., the use of stem cells that do not fully differentiate and do not properly integrate into the heart tissue), some of these failures could be attributed to the evolutional differences between rodents and humans. Further, mouse MI models do not necessarily recapitulate clinical treatment routines seen in patients. For example, a permanent Left Artery Descending (LAD) ligation protocol for MI, does not include timely reperfusion of the infarcted area, unlike the clinical attempt to reopen the infarct-related artery by percutaneous coronary intervention (PCI). Inherent differences in heart vasculature anatomy and proportions as well as composition of the immune system between rodents and humans can lead to the different magnitude of the ischemia-reperfusion injury. Finally, in the mouse model researchers use highly invasive trajectories, i. e. thoracotomy, that are not clinically relevant and would create excessive damage to the patient. Therefore, examining the effect of a potential therapy in a clinically relevant MI model, where the mode of application, dosage and regiment are driven by patient care, may help to bridge the gap from bench to bedside.

The ECM protein Agrin can promote heart regeneration in a mouse model of myocardial infarction^{s} (see also WO2017/072772). Agrin induces CM cell cycle re-entry and division in vitro and is required for the full regenerative capacity of neonatal mouse hearts. In vitro, recombinant Agrin promotes the division of mouse and human iPSC-derived CM via mechanisms that involve CM dedifferentiation and downstream signals mediated by Yap and ERK signaling pathways. In vivo, a single administration of Agrin promotes cardiac regeneration in adult mice after MI⁵.

### SUMMARY OF THE INVENTION

According to an aspect of some embodiments of the present invention there is provided a method of treating an ischemic heart disease in a subject in need thereof, the method comprising administering to the subject a therapeutically effective amount of Agrin in an anterograde intracoronary manner, thereby treating the ischemic heart disease in the subject.

According to an aspect of some embodiments of the present invention there is provided a therapeutically effective amount of Agrin for use in administration in an anterograde intracoronary manner to a subject in need thereof for the treatment of an ischemic heart disease.

According to some embodiments of the invention, the ischemic heart disease is selected from the group consisting of acute myocardial infarction (AMI), myocardial infarction (MI) and Chronic heart failure (CHF).

According to some embodiments of the invention, the therapeutically effective amount comprises a single administration.

According to some embodiments of the invention, the therapeutically effective amount comprises a repeated administration.

According to some embodiments of the invention, the repeated administration comprises at least 2 administrations. According to some embodiments of the invention, a first administration of the at least 2 administrations is immediately after diagnosis of the ischemic heart disease and a second administration of the two administrations is within 96 hours from the diagnosis.

According to some embodiments of the invention, a first administration of the at least 2 administrations is immediately after diagnosis of the ischemic heart disease and a second administration of the two administrations is within 48-96 hours from the diagnosis.

According to some embodiments of the invention, the therapeutically effective amount is 20-50 μg/Kg.

According to some embodiments of the invention, the Agrin is an Agrin peptide capable of inducing cardiomyocyte proliferation.

According to some embodiments of the invention, the Agrin is not a part of a fusion polypeptide.

According to some embodiments of the invention, the Agrin is in a soluble form.

According to some embodiments of the invention, the Agrin peptide comprises a laminin G-like 1 domain (G1) and a laminin G-like 2 domain (G2).

According to some embodiments of the invention, the Agrin peptide comprises a laminin G-like 1 domain (G1) and a laminin G-like 2 domain (G2) and is devoid of a laminin G-like 3 domain (G3).

According to some embodiments of the invention, the Agrin peptide is 150-600 amino acids long.

According to some embodiments of the invention, the Agrin peptide is 200-600 amino acids long.

According to some embodiments of the invention, the Agrin peptide is 200-520 amino acids long.

According to some embodiments of the invention, the Agrin peptide is 400-520 amino acids long.

According to some embodiments of the invention, the Agrin peptide is 300-520 amino acids long.

According to some embodiments of the invention, the Agrin peptide is human Agrin.

According to some embodiments of the invention, the Agrin comprises a fragment of human Agrin.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-D compare different Agrin delivery methods into the infarcted pig's heart. (FIGS. 1A-B) Scheme describing the delivery experiments: (FIG. 1A) Pigs were subjected to MI by balloon occlusion of the LAD (after the first diagonal) for 60′. During reperfusion, Agrin (33 μg/Kg) was introduced in one of three trajectories: anterograde (n=3), retrograde (n=2) or intramyocardial (n=2). After an additional 60′, hearts were harvested. (FIG. 1B) To allow for spatial analysis of Agrin distribution in the infarcted heart, hearts were sectioned transversely into 5 sections, and each respective section was further dissected into 4 (apex) or 8 segments. Each segment was numbered and annotated as infarct (colored in black), border zone (bordering an infarcted segment) or control. (FIGS. 1C-D) Assessment of Agrin distribution in the different segments of the infracted hearts. Animals were treated and sectioned as described in FIGS. 1A-B. Protein was extracted from each section, and the protein extracts underwent anti-His IP to enrich for Agrin. Agrin amount was determined by Western blot. (FIG. 1C) Representative Western blot image comparing the amount of Agrin using all three trajectories in three segments: control (26), infarct (31), border zone (32). 5 ng of recombinant Agrin served as positive control (+); Ant-Antrograde, Ret-retrograde, i.m.—intramyocardial. (FIG. 1D) Color coded heat map of the average intensity at each segment for each trajectory; WB Agrin intensity of each segment were normalized to the positive control, averaged and color coded (red-high Agrin, blue-no Agrin, wight-not assayed). Each mode of delivery is presented in a different row;

FIGS. 2A-F show that heart function is improved in Agrin treated pigs post MI. (FIG. 2A) Scheme describing the experimental plan of the pig MI model. Baseline measurements of heart function were acquired, using MRI, PV loop, fluoroscopy and blood samples. MI and Agrin treatment were induced as described in FIG. 2A, using the antegrade method. Saline was used as control. 3 days post MI, all animals were subjected to fluoroscopy and PV-loop measurements, and several animals underwent a second Agrin treatment, 3 days post MI. Animals were monitored by MRI 25 days post MI. Heart function was measured 28 days post MI by fluoroscopy and PV-loop. Animals were then sacrificed and hearts were explanted for histological analysis. (Saline n=8, Single (Agrin 1 dose) Agrin treatment n=6, Dual (Agrin 2 doses) Agrin treatment n=5). (FIGS. 2C-D) Bar graphs depicting the EF changes, derived from fluoroscopy analysis; (FIG. 2B) Bar graph depicting EF values at baseline, 3 and 28 days post MI. (FIG. 2C) Bar graph describing the reduction in EF in the different treatments at experimental end point. (FIG. 2D) Bar graph demonstrating the changes in stroke volumes at end point, as determined by MRI analysis; (FIG. 2E-F) Bar graphs showing the change LVEDP, as measured by PV-loop; (FIG. 2E) Bar graph depicting LVEDP values at baseline, 3 and 28 days post MI. (FIG. 2F) Bar graph demonstrating the changes in LVEDP in the different treatments at experimental end point; *-p<0.05,***-p<0.001.

FIGS. 3A-D show that heart scarring is improved in Agrin treated pigs post MI. Scar of the infarcted heart was measured using several methodologies; (FIG. 3A) upper panel: representative images of heart sections after TTC staining (white represents scar tissue, red represents viable myocardium); lower panel: Same images with a graphic mask depicting healthy tissue (red) and scar (black). (FIG. 3B) Bar graph depicting the area at risk (AAR) as percent of left ventricle wall. The AAR was measured by applying TTC to the occlusion site in the LAD, and measuring the perfused area. AAR was similar in all groups, indicating similar LAD occlusions. (FIG. 3C) Bar graph describing the scar tissue as a percent of the left ventricle wall, derived from TTC image analysis. (FIG. 3D) Bar graph describing the scar tissue as retention of contrast agent (late enhancement, acquired by MRI at 25 days post MI); ns-non significant *-p<0.05,**-p<0.01.

FIGS. 4A-B show that heart myocardium contraction is improved in Agrin treated hearts. Subendocardial segment shortening (SES) was measured at experimental endpoint in (FIG. 4A) border zone and (FIG. 4B) infarct regions of the injured hearts (presented as % of the nonischemic Cx region) at rest, 120/min and 150/min atrial pacing (Saline n=4, Single (Agrin 1 dose) Agrin treatment n=6, Dual (Agrin 2 doses) Agrin treatment n=2).

FIGS. 5A-D show that Agrin improves heart function in a rodent model of chronic heart failure (CHF). (FIG. 5A) Scheme describing the experimental plan of the Rat CHF model. Baseline measurements of heart function were acquired using ultrasound echocardiography. MI was induced by permanent LAD ligation, and MI severity was assessed by Echocardiography 2 days post MI. After randomization, animals were treated with intramyocardial injection of Agrin or Saline (control) 28 days post MI. Heart function was measured again, 35 days post treatment (63days post MI). (FIG. 5B-C) Bar graphs depicting the EF changes, derived from Echocardiography analysis; (FIG. 5B) Bar graph depicting EF values at baseline, 21 and 63days post MI. (FIG. 5C) Bar graph describing the reduction in EF in the different treatments at experimental end point. (FIG. 5D) Stacked column graph describing the distribution of EF modulation in the different treatment groups (Saline n=12, Agrin treatment n=14); ns-non significant, *-p<0.05, **-p<0.01.

FIGS. 6A-D show that Agrin prevents remodeling of the heart post MI. (FIGS. 6A-B) Heart weight to body weight ratio (HW/BW) (FIG. 6A) Representative images of saline (Ctrl) and Agrin treated hearts. (FIG. 6B) Bar graphshowing the HW/BW at experimental end stage. (FIGS. 6C-D). CM size was deduced from WGA staining image analysis of several sections of the treated hearts. (FIG. 6C) Representative images of heart sections stained with WGA. (FIG. 6D) Bar graph showing the differences in CM average size at the experimental end point.

### DESCRIPTION OF SPECIFIC EMBODIMENTS OF THE INVENTION

The present invention, in some embodiments thereof, relates to methods of treating ischemic heart diseases.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Ischemic heart diseases are classified among the leading cause of death and reduced quality of life worldwide. Unfortunately, many of the promising therapeutic approaches, aiming at ischemia-reperfusion injury and postischemic adverse remodeling of the heart, did not translate into successful clinical application, emphasizing the need to conduct large-animal clinically-relevant models. Inventors of embodiments described herein have previously shown (also described in WO2017/072772) that the ECM protein Agrin promotes cardiac regeneration following myocardial infarction in mice.

Whilst reducing embodiments of the invention to practice, the present inventors have performed a series of experiments conducted in a large animal model of acute myocardial infarction in pigs and found that anterograde intracoronary delivery of the protein to be an efficient and clinically applicable method of Agrin administration into injured heart tissue. This method allowed for specific targeted delivery of the protein to the infarct and border zone regions of injured hearts. Finally, applying this method in the pig model revealed significant protective and regenerative effects of Agrin, and suggests its use in preventing heart failure. The present findings support the use of Agrin as a potential therapy for human ischemic heart and provide beneficial dosage and delivery regimen.

Thus, according to an aspect of the invention there is provided a method of treating an ischemic heart disease in a subject in need thereof, the method comprising administering to the subject a therapeutically effective amount of Agrin in an anterograde intracoronary manner, thereby treating the ischemic heart disease in the subject.

According to an aspect of the invention there is provided a therapeutically effective amount of Agrin for use in administration in an anterograde intracoronary manner to a subject in need thereof for the treatment of an ischemic heart disease.

As used herein “a cardiomyocyte” or “cardiomyocytes” (abbreviated as, CM, CMs), also known as myocardiocytes or cardiac myocytes, are the muscle cells (myocytes) that make up the cardiac muscle. The term refers to cardiomyocytes of any species including mammalian, e.g., human at any stage of development. According to a specific embodiment, the cardiomyocyte is a neonatal CM (e.g., for human up 6 months after birth). According to a specific embodiment, the cardiomyocyte is an adult cardiomyocyte (e.g., for human at least 16-18 years after birth).

Thus, according to a specific embodiment, the cardiomyocytes are of a subject having a heart disease.

According to a specific embodiment, the cardiomyocytes may be naturally occurring. According to a specific embodiment, the CMs have been ex-vivo differentiated into cardiomyocytes (e.g., from pluripotent stem cells e.g., embryonic stem cells (hESCs) and induced pluripotent stem cells (iPSCs)). Methods of differentiating stem cells into CMs are well known in the art. For example, an iPSC can be co-cultured with visceral endoderm-like cells (see, e.g., Mummery et al. (2003) Circulation 107:2733). An iPS cell can also be induced to undergo cardiomyogenesis without co-culture with a feeder cell or other cell. For example, as described in U.S. Pat. No. 7,297,539. The CMs may be fully differentiated when contacted with the agent (e.g., Agrin). According to another embodiment, the cells are committed to the cardiac lineage and the agent (e.g., Agrin) is added to the culture during or following the differentiation process.

According to a specific embodiment, the cardiomyocytes are human CMs.

According to a specific embodiment, the CMs are a cell-line.

According to a specific embodiment, the CMs are primary CMs.

According to a specific embodiment, the Agrin is capable of inducing CM proliferation.

As used herein the term “inducing proliferation” refers to an increase in CM proliferation which is statistically significant (as compared to untreated cells of the same origin and developmental stage) and is a result of contacting the cardiomyocytes with the agent e.g., Agrin.

According to an additional or an alternative embodiment the Agrin is capable of inducing immune modulation by which increasing cardiomyocyte survival, anti-inflammatory and/or anti fibrotic effects and/or tissue protective effect.

As used herein “immune modulation” refers to induced changes in gene expression (e.g., RNA as determined by RNA-Seq) of canonical pathway genes—and/or upstream regulators.

As used herein the term “Agrin” refers to the protein product of the AGRN gene. The term is meant to include polynucleotide sequences encoding Agrin or expression products as RNA or a protein.

According to a specific embodiment, the Agrin is human Agrin.

According to a specific embodiment, Agrin refers to the full-length naturally occurring Agrin (e.g., human). However, according to a specific embodiment, the Agrin is an Agrin peptide (which is typically more suitable for use in therapy). An “Agrin peptide” refers to an Agrin peptide which is shorter than the full-length Agrin (e.g., in the case of human Agrin shorter than the 2068/2045 amino acids which make up the full length human Agrins) and is capable of inducing proliferation of cardiomyocytes (e.g., at least in vitro such as described in WO2017/072772). According to a specific embodiment the Agrin peptide is provided in a soluble form.

According to a specific embodiment and as mentioned, the Agrin peptide is from human Agrin NP_001292204 (SEQ ID NO: 4) or NP_940978 (SEQ ID NO: 5) or Uniprot 000468 SEQ ID NO: 38.

According to a specific embodiment, the Agrin peptide is of a human ortholog e.g., NP_786930 (SEQ ID NO: 6).

It will be appreciated that the present teachings contemplate the treatment of one species (e.g., human) with an Agrin peptide of a second species (e.g., rat) as long as they exhibit the desired activity (i.e., induced CM proliferation), protective and/or regenerative on the treated subject/cells.

According to a specific embodiment, the Agrin peptide comprises a Laminin G-like 1 (G1) domain, a Laminin G-like 2 (G2) domain and Laminin G-like 3 (G3) domain.

According to a specific embodiment, the Agrin peptide comprises a Laminin G-like 2 (G2) domain and Laminin G-like 1 (G1) domain.

Accordingly there is provided an Agrin peptide is typically 200-600 amino acids in length or as further described hereinbelow.

According to a specific embodiment, such an Agrin peptide promotes heart regeneration. According to a specific embodiment, the Agrin peptide is 20-100 kDa. According to a specific embodiment, the Agrin peptide is 50-100 kDa. According to a specific embodiment, the Agrin peptide is 80-100 kDa.

According to a specific embodiment, the Agrin peptide is 80-90 kDa.

Agrin peptides are commercially available from R&D systems e.g., 6624-AG, 550-AG or 550-AG/CF.

According to a specific embodiment the Agrin is recombinant Agrin (rAgrin) 6624-AG, R&D biosystems, USA).

According to a specific embodiment, the Agrin is not a part of a fusion polypeptide where the Agrin is serving as a targeting moiety for the delivery of a therapeutically effective peptide.

According to a specific embodiment, the Agrin is provided in a soluble form. Accordingly, the Agrin is not part or attached to an extracellular matrix composition.

Methods of determining CM proliferation are well known in the art, and include, but are not limited to, manual cell counting, MTT assay and a thymidine incorporation assay. According to some embodiments both ascertaining the nature of the cells as well as determining their proliferation are done.

For example, in some embodiments, the presence of proliferative cardiomyocytes is validated by confirming expression of at least one cardiomyocyte-specific marker produced by the cell. For example, the cardiomyocytes express cardiac transcription factors, sarcomere proteins, and gap junction proteins. Suitable cardiomyocyte-specific proteins include, but are not limited to, cardiac troponin I, cardiac troponin-C, tropomyosin, caveolin-3, GATA-4, myosin heavy chain, myosin light chain-2a, myosin light chain-2v, ryanodine receptor, and atrial natriuretic factor.

As another example, in some embodiments, cardiomyocytes are ascertained by detecting responsiveness to pharmacological agents such as beta-adrenergic agonists (e.g., isoprenaline), adrenergic beta-antagonists (e.g., esmolol), cholinergic agonists (e.g., carbochol), and the like.

Alternatively or additionally, validating the nature of the CMs is done by detecting electrical activity of the cells. Electrical activity can be measured by various methods, including extracellular recording, intracellular recording (e.g., patch clamping), and use of voltage-sensitive dyes. Such methods are well known to those skilled in the art.

The term “peptide” as used herein encompasses native peptides (either degradation products, synthetically synthesized peptides or recombinant peptides) and peptidomimetics (typically, synthetically synthesized peptides), as well as peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder. According to a specific embodiment, the peptide (or polypeptide) is a recombinant product (i.e., of recombinant DNA technology). According to a specific embodiment, the Agrin is above 95% pure (e.g., no other active ingredient proteins are present in the formulation).

Peptide bonds (—CO—NH—) within the peptide may be substituted, for example, by N-methylated amide bonds (—N(CH3)—CO—), ester bonds (—C(═O)—O—), ketomethylene bonds (—CO—CH2—), sulfinylmethylene bonds (—S(═O)—CH2—), α-aza bonds (—NH—N(R)—CO—), wherein R is any alkyl (e.g., methyl), amine bonds (—CH2—NH—), sulfide bonds (—CH2—S—), ethylene bonds (—CH2—CH2—), hydroxyethylene bonds (—CH(OH)—CH2—), thioamide bonds (—CS—NH—), olefinic double bonds (—CH═CH—), fluorinated olefinic double bonds (—CF═CH—), retro amide bonds (—NH—CO—), peptide derivatives (—N(R)—CH2—CO—), wherein R is the “normal” side chain, naturally present on the carbon atom.

These modifications can occur at any of the bonds along the peptide chain and even at several (2-3) bonds at the same time.

Natural aromatic amino acids, Trp, Tyr and Phe, may be substituted by non-natural aromatic amino acids such as 1,2,3,4-tetrahydroisoquinoline-3-carboxylic acid (Tic), naphthylalanine, ring-methylated derivatives of Phe, halogenated derivatives of Phe or O-methyl-Tyr.

The peptides of some embodiments of the invention may also include one or more modified amino acids or one or more non-amino acid monomers (e.g. fatty acids, complex carbohydrates etc).

The term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D1and L-amino acids.

Tables 1 and 2 below list naturally occurring amino acids (Table 1), and non-conventional or modified amino acids (e.g., synthetic, Table 2) which can be used with some embodiments of the invention.

TABLE 1

| | Three-Letter | One-letter |
|---|---|---|
| Amino Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid as above | Xaa | X |

TABLE 2

| Non-conventional | | Non-conventional | |
|---|---|---|---|
| amino acid | Code | amino acid | Code |
| ornithine | Orn | hydroxyproline | Hyp |
| α-aminobutyric acid | Abu | aminonorbornyl-carboxylate | Norb |
| D-alanine | Dala | aminocyclopropane-carboxylate | Cpro |
| D-arginine | Darg | N-(3-guanidinopropyl)glycine | Narg |
| D-asparagine | Dasn | N-(carbamylmethyl)glycine | Nasn |
| D-aspartic acid | Dasp | N-(carboxymethyl)glycine | Nasp |
| D-cysteine | Dcys | N-(thiomethyl)glycine | Ncys |
| D-glutamine | Dgln | N-(2-carbamylethyl)glycine | Ngln |
| D-glutamic acid | Dglu | N-(2-carboxyethyl)glycine | Nglu |
| D-histidine | Dhis | N-(imidazolylethyl)glycine | Nhis |
| D-isoleucine | Dile | N-(1-methylpropyl)glycine | Nile |
| D-leucine | Dleu | N-(2-methylpropyl)glycine | Nleu |
| D-lysine | Dlys | N-(4-aminobutyl)glycine | Nlys |
| D-methionine | Dmet | N-(2-methylthioethyl)glycine | Nmet |
| D-ornithine | Dorn | N-(3-aminopropyl)glycine | Norn |
| D-phenylalanine | Dphe | N-benzylglycine | Nphe |
| D-proline | Dpro | N-(hydroxymethyl)glycine | Nser |
| D-serine | Dser | N-(1-hydroxyethyl)glycine | Nthr |
| D-threonine | Dthr | N-(3-indolylethyl) glycine | Nhtrp |
| D-tryptophan | Dtrp | N-(p-hydroxyphenyl)glycine | Ntyr |
| D-tyrosine | Dtyr | N-(1-methylethyl)glycine | Nval |
| D-valine | Dval | N-methylglycine | Nmgly |
| D-N-methylalanine | Dnmala | L-N-methylalanine | Nmala |
| D-N-methylarginine | Dnmarg | L-N-methylarginine | Nmarg |
| D-N-methylasparagine | Dnmasn | L-N-methylasparagine | Nmasn |
| D-N-methylasparatate | Dnmasp | L-N-methylaspartic acid | Nmasp |
| D-N-methylcysteine | Dnmcys | L-N-methylcysteine | Nmcys |
| D-N-methylglutamine | Dnmgln | L-N-methylglutamine | Nmgln |
| D-N-methylglutamate | Dnmglu | L-N-methylglutamic acid | Nmglu |
| D-N-methylhistidine | Dnmhis | L-N-methylhistidine | Nmhis |
| D-N-methylisoleucine | Dnmile | L-N-methylisolleucine | Nmile |
| D-N-methylleucine | Dnmleu | L-N-methylleucine | Nmleu |
| D-N-methyllysine | Dnmlys | L-N-methyllysine | Nmlys |
| D-N-methylmethionine | Dnmmet | L-N-methylmethionine | Nmmet |
| D-N-methylornithine | Dnmorn | L-N-methylornithine | Nmorn |
| D-N-methylphenylalanine | Dnmphe | L-N-methylphenylalanine | Nmphe |
| D-N-methylproline | Dnmpro | L-N-methylproline | Nmpro |
| D-N-methylserine | Dnmser | L-N-methylserine | Nmser |
| D-N-methylthreonine | Dnmthr | L-N-methylthreonine | Nmthr |
| D-N-methyltryptophan | Dnmtrp | L-N-methyltryptophan | Nmtrp |
| D-N-methyltyrosine | Dnmtyr | L-N-methyltyrosine | Nmtyr |
| D-N-methylvaline | Dnmval | L-N-methylvaline | Nmval |
| L-norleucine | Nle | L-N-methylnorleucine | Nmnle |
| L-norvaline | Nva | L-N-methylnorvaline | Nmnva |
| L-ethylglycine | Etg | L-N-methyl-ethylglycine | Nmetg |
| L-t-butylglycine | Tbug | L-N-methyl-t-butylglycine | Nmtbug |
| L-homophenylalanine | Hphe | L-N-methyl-homophenylalanine | Nmhphe |
| α-naphthylalanine | Anap | N-methyl-α-naphthylalanine | Nmanap |
| penicillamine | Pen | N-methylpenicillamine | Nmpen |
| γ-aminobutyric acid | Gabu | N-methyl-γ-aminobutyrate | Nmgabu |
| cyclohexylalanine | Chexa | N-methyl-cyclohexylalanine | Nmchexa |
| cyclopentylalanine | Cpen | N-methyl-cyclopentylalanine | Nmcpen |
| α-amino-α- | Aabu | N-methyl-α-amino-α- | Nmaabu |
| methylbutyrate | | methylbutyrate | |
| α-aminoisobutyric acid | Aib | N-methyl-α-aminoisobutyrate | Nmaib |
| D-α-methylarginine | Dmarg | L-α-methylarginine | Marg |
| D-α-methylasparagine | Dmasn | L-α-methylasparagine | Masn |
| D-α-methylaspartate | Dmasp | L-α-methylaspartate | Masp |
| D-α-methylcysteine | Dmcys | L-α-methylcysteine | Mcys |
| D-α-methylglutamine | Dmgln | L-α-methylglutamine | Mgln |
| D-α-methyl glutamic acid | Dmglu | L-α-methylglutamate | Mglu |
| D-α-methylhistidine | Dmhis | L-α-methylhistidine | Mhis |
| D-α-methylisoleucine | Dmile | L-α-methylisoleucine | Mile |
| D-α-methylleucine | Dmleu | L-α-methylleucine | Mleu |
| D-α-methyllysine | Dmlys | L-α-methyllysine | Mlys |
| D-α-methylmethionine | Dmmet | L-α-methylmethionine | Mmet |
| D-α-methylornithine | Dmorn | L-α-methylornithine | Morn |
| D-α-methylphenylalanine | Dmphe | L-α-methylphenylalanine | Mphe |
| D-α-methylproline | Dmpro | L-α-methylproline | Mpro |
| D-α-methylserine | Dmser | L-α-methylserine | Mser |
| D-α-methylthreonine | Dmthr | L-α-methylthreonine | Mthr |
| D-α-methyltryptophan | Dmtrp | L-α-methyltryptophan | Mtrp |
| D-α-methyltyrosine | Dmtyr | L-α-methyltyrosine | Mtyr |
| D-α-methylvaline | Dmval | L-α-methylvaline | Mval |
| N-cyclobutylglycine | Ncbut | L-α-methylnorvaline | Mnva |
| N-cycloheptylglycine | Nchep | L-α-methylethylglycine | Metg |
| N-cyclohexylglycine | Nchex | L-α-methyl-t-butylglycine | Mtbug |
| N-cyclodecylglycine | Ncdec | L-α-methyl-homophenylalanine | Mhphe |
| N-cyclododecylglycine | Ncdod | α-methyl-α-naphthylalanine | Manap |
| N-cyclooctylglycine | Ncoct | α-methylpenicillamine | Mpen |
| N-cyclopropylglycine | Ncpro | α-methyl-γ-aminobutyrate | Mgabu |
| N-cycloundecylglycine | Ncund | α-methyl-cyclohexylalanine | Mchexa |
| N-(2-aminoethyl)glycine | Naeg | α-methyl-cyclopentylalanine | Mcpen |
| N-(2,2-diphenylethyl)glycine | Nbhm | N-(N-(2,2-diphenylethyl) | Nnbhm |
| | | carbamylmethyl-glycine | |
| N-(3,3-diphenylpropyl)glycine | Nbhe | N-(N-(3,3-diphenylpropyl) | Nnbhe |
| | | carbamylmethyl-glycine | |
| 1-carboxy-1-(2,2-diphenyl- | Nmbc | 1,2,3,4-tetrahydroisoquinoline-3- | Tic |
| ethylamino)cyclopropane | | carboxylic acid | |
| phosphoserine | pSer | phosphothreonine | pThr |
| phosphotyrosine | pTyr | O-methyl-tyrosine | |
| 2-aminoadipic acid | | hydroxylysine | |

The peptides of some embodiments of the invention are preferably utilized in a linear form, although it will be appreciated that in cases where cyclicization does not severely interfere with peptide characteristics, cyclic forms of the peptide can also be utilized.

Since the present peptides are preferably utilized in therapeutics or diagnostics which require the peptides to be in soluble form, the peptides of some embodiments of the invention preferably include one or more non-natural or natural polar amino acids, including but not limited to serine and threonine which are capable of increasing peptide solubility due to their hydroxyl-containing side chain.

The peptides of some embodiments of the invention are preferably utilized in a linear form, although it will be appreciated that in cases where cyclicization does not severely interfere with peptide characteristics, cyclic forms of the peptide can also be utilized.

According to a specific embodiment, Agrin peptide is 150-600 amino acids long.

According to a specific embodiment, Agrin is 200-600 amino acids long.

According to a specific embodiment, Agrin peptide is 200-520 amino acids long.

According to a specific embodiment, Agrin peptide is 400-520 amino acids long.

According to a specific embodiment, Agrin peptide is 300-520 amino acids long.

It will be appreciated that the proteinaceous agents of some embodiments of the invention, can also utilize functional homologues which exhibit the desired activity (i.e., induced proliferation of CMs). Such homologues can be, for example, at least, 60%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the human sequence e.g., human Agrin e.g., SEQ ID NO: 4, 5, 7 or 8, as determined using the BestFit software of the Wisconsin sequence analysis package, utilizing the Smith and Waterman algorithm, where gap weight equals 50, length weight equals 3, average match equals 10 and average mismatch equals −9.

The peptides of some embodiments of the invention may be synthesized by any techniques that are known to those skilled in the art of peptide synthesis. For solid phase peptide synthesis, a summary of the many techniques may be found in J. M. Stewart and J. D. Young, Solid Phase Peptide Synthesis, W. H. Freeman Co. (San Francisco), 1963 and J. Meienhofer, Hormonal Proteins and Peptides, vol. 2, p. 46, Academic Press (New York), 1973. For classical solution synthesis see G. Schroder and K. Lupke, The Peptides, vol. 1, Academic Press (New York), 1965.

In general, these methods comprise the sequential addition of one or more amino acids or suitably protected amino acids to a growing peptide chain. Normally, either the amino or carboxyl group of the first amino acid is protected by a suitable protecting group. The protected or derivatized amino acid can then either be attached to an inert solid support or utilized in olution by adding the next amino acid in the sequence having the complimentary (amino or carboxyl) group suitably protected, under conditions suitable for forming the amide linkage. The protecting group is then removed from this newly added amino acid residue and the next amino acid (suitably protected) is then added, and so forth. After all the desired amino acids have been linked in the proper sequence, any remaining protecting groups (and any solid support) are removed sequentially or concurrently, to afford the final peptide compound.

Alternatively, the peptides are produced using recombinant DNA technology.

A variety of prokaryotic or eukaryotic cells can be used as host-expression systems to express the polypeptides/peptides of some embodiments of the invention. These include, but are not limited to, microorganisms, such as bacteria transformed with a recombinant bacteriophage DNA, plasmid DNA or cosmid DNA expression vector containing the coding sequence; yeast transformed with recombinant yeast expression vectors containing the coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the coding sequence. Mammalian expression systems can also be used to express the polypeptides of some embodiments of the invention.

For the sake of simplicity Agrin and the agent are collectively referred to herein as “an agent” or “agents”, although it should be appreciated that each possibility of an agent represents a separate embodiment of the present invention.

According to a specific embodiment, the proteinaceous agent can be attached (or conjugated) to non-proteinaceous moieties which increase their bioavailability and half-life in the circulation.

The phrase “non-proteinaceous moiety” as used herein refers to a molecule not including peptide bonded amino acids that is attached to the above-described proteinaceous agents. Exemplary non-proteinaceous and preferably non-toxic moieties which may be used according to the present teachings include, but are not limited to, polyethylene glycol (PEG), Polyvinyl pyrrolidone (PVP), poly(styrene comaleic anhydride) (SMA), and divinyl ether and maleic anhydride copolymer (DIVEMA).

Such a molecule is highly stable (resistant to in-vivo proteolytic activity probably due to steric hindrance conferred by the non-proteinaceous moiety) and may be produced using common solid phase synthesis methods which are inexpensive and highly efficient, as further described hereinbelow. However, it will be appreciated that recombinant techniques may still be used, whereby the recombinant peptide product is subjected to in-vitro modification (e.g., PEGylation).

Thus, such non-proteinaceous non-toxic moieties may also be attached to the above mentioned agents to promote stability and possibly solubility of the molecules.

Bioconjugation of such a non-proteinaceous moiety (such as PEGylation) can confer the proteins amino acid sequence with stability (e.g., against protease activities) and/or solubility (e.g., within a biological fluid such as blood, digestive fluid) while preserving its biological activity and prolonging its half-life.

Bioconjugation is advantageous particularly in cases of therapeutic proteins which exhibit short half-life and rapid clearance from the blood. The increased half-lives of bioconjugated proteins in the plasma results from increased size of protein conjugates (which limits their glomerular filtration) and decreased proteolysis due to polymer steric hindrance. Generally, the more polymer chains attached per peptide, the greater the extension of half-life. However, measures are taken not to reduce the specific activity of the protein of the present invention (e.g., CM proliferation).

Bioconjugation of the proteinaceous agent with PEG (i.e., PEGylation) can be effected using PEG derivatives such as N-hydroxysuccinimide (NHS) esters of PEG carboxylic acids, monomethoxyPEG2-NHS, succinimidyl ester of carboxymethylated PEG (SCM-PEG), benzotriazole carbonate derivatives of PEG, glycidyl ethers of PEG, PEG p-nitrophenyl carbonates (PEG-NPC, such as methoxy PEG-NPC), PEG aldehydes, PEG-orthopyridyl-disulfide, carbonyldimidazol-activated PEGs, PEG-thiol, PEG-maleimide. Such PEG derivatives are commercially available at various molecular weights [See, e.g., Catalog, Polyethylene Glycol and Derivatives, 2000 (Shearwater Polymers, Inc., Huntsvlle, Ala.)]. If desired, many of the above derivatives are available in a monofunctional monomethoxyPEG (mPEG) form. In general, the PEG added to the anti HER3 antibody amino acid sequence of the present invention should range from a molecular weight (MW) of several hundred Daltons to about 100 kDa (e.g., between 3-30 kDa). Larger MW PEG may be used, but may result in some loss of yield of PEGylated peptides. The purity of larger PEG molecules should be also watched, as it may be difficult to obtain larger MW PEG of purity as high as that obtainable for lower MW PEG. It is preferable to use PEG of at least 85% purity, and more preferably of at least 90% purity, 95% purity, or higher. PEGylation of molecules is further discussed in, e.g., Hermanson, Bioconjugate Techniques, Academic Press San Diego, Calif. (1996), at Chapter 15 and in Zalipsky et al., “Succinimidyl Carbonates of Polyethylene Glycol,” in Dunn and Ottenbrite, eds., Polymeric Drugs and Drug Delivery Systems, American Chemical Society, Washington, D.C. (1991).

The ability to induce CM proliferation renders the present teachings particularly suitable for the treatment of heart diseases where there is damage to the cardiac tissue or there is a risk for such damage.

The term “treating” refers to inhibiting, preventing or arresting the development of a pathology (i.e., heart disease, disorder or condition, e.g., ischemic heart disease) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “preventing” refers to keeping a disease, disorder or condition from occurring in a subject who may be at risk for the disease, but has not yet been diagnosed as having the disease.

As used herein, the term “subject” includes mammals, preferably human beings at any age that suffer from the pathology. Preferably, this term encompasses individuals who are at risk to develop the pathology.

According to a specific embodiment, the heart disease is an ischemic heart disease.

An ischemic heart disease refers to a lack of oxygen flow to the heart or portion thereof, resulting in myocardial ischemic damage. As used herein, the phrase myocardial ischemic damage includes damage caused by reduced blood flow to the myocardium. Non-limiting examples of causes of an ischemic heart disease and myocardial ischemic damage include: decreased aortic diastolic pressure, increased intraventricular pressure and myocardial contraction, coronary artery stenosis (e.g., coronary ligation, fixed coronary stenosis, acute plaque change (e.g., rupture, hemorrhage), coronary artery thrombosis, vasoconstriction), aortic valve stenosis and regurgitation, and increased right atrial pressure. Non-limiting examples of adverse effects of myocardial ischemia and myocardial ischemic damage include myocyte damage (e.g., myocyte cell loss, myocyte hypertrophy, myocyte cellular hyperplasia), angina (e.g., stable angina, variant angina, unstable angina, sudden cardiac death), myocardial infarction, and congestive heart failure. Damage due to myocardial ischemia may be acute or chronic, and consequences may include scar formation, cardiac remodeling, cardiac hypertrophy, wall thinning, dilatation, and associated functional changes. The existence and etiology of acute or chronic myocardial damage and/or myocardial ischemia may be diagnosed using any of a variety of methods and techniques well known in the art including, e.g., non-invasive imaging (e.g., MRI, echocardiography), angiography, stress testing, assays for cardiac-specific proteins such as cardiac troponin, and evaluation of clinical symptoms. These methods and techniques as well as other appropriate techniques may be used to determine which subjects are suitable candidates for the treatment methods described herein.

According to a specific embodiment, the ischemic heart disease in the present invention includes, for example, coronary arteriosclerosis, acute myocardial infarction (AMI), myocardial infarction (MI), old MI, angina pectoris (AP) including stable angina, unstable angina, and effort angina, ischemic cardiomyopathy, heart failure, and other disease which causes necrosis of heart muscle that results from prolonged ischemia. As necrosis of heart muscle progresses, the damaged myocardiac tissue are replaced with fibrous tissue, thickness of the myocardial wall in the infarct zone gets thinner, and the cardiac inner cavity dilates, therefore cardiac function such as contractility deteriorates and results in heart failure.

Coronary arteriosclerosis is characterized by arteriosclerosis in the coronary artery that supplies nutrients to the heart. Angina pectoris is characterized by attacks of chest pain caused by impaired blood flow in the coronary artery. Myocardial infarction is characterized by myocardial necrosis caused by impaired blood flow in the coronary artery and by fatal complications coming therewith such as arrhythmia, cardiac failure, cardiac rupture, and pump failure. Impaired blood flow to the heart, a vital organ, is an essential characteristic of these ischemic heart diseases.

“Post-infarction myocardial remodeling” refers to a series of changes such as the hypertrophy of myocardial cells at non-infarction sites, increase in interstitial tissue (extracellular matrix), and the dilation of cardiac lumens, which occur in compensation for reduced cardiac function caused by thickening at infarction sites after myocardial infarction. Since long-term prognosis after myocardial infarction is correlated with the degree of left ventricular dysfunction, the suppression of myocardial remodeling is important for maintaining and conserving the function of the left ventricle.

According to a specific embodiment the ischemic heart disease is selected from the group consisting of acute myocardial infarction (AMI), myocardial infarction (MI), Chronic heart failure (CHF).

As used herein “anterograde intracoronary administration” refers to injection into the blocked coronary artery with a standard catheter (with the blood flow using an autoperfusion balloon angioplasty catheter). In the case of ongoing ischemic disease, the injection is performed in the process of clinical reperfusion, injecting via the same catheter used for reperfusion.

The amount of a composition to be administered will, of course, be dependent on the subject being treated, the severity of the affliction, the manner of administration, the judgment of the prescribing physician, etc.

According to a specific embodiment the therapeutically effective amount comprises a single administration.

Typically, such administration is immediate is immediately after diagnosis of the ischemic heart disease e.g., up to 12 hours following diagnosis and/or concomitantly with treatment e.g., angioplasty performed for reperfusion of the ischemic heart. Administration will be done “over the wire” into the affected regions of the ischemic heart using the same catheter directed at autoperfusion.

Agrin may be administered one or several times following diagnosis:

Thus according to some embodiments of the invention the therapeutically effective amount comprises a repeated administration.

According to a specific embodiment, the repeated administration comprises at least 2 administrations.

According to a specific embodiment, the first administration of the at least 2 administrations is immediately after diagnosis e.g., up to 12 hours following diagnosis and/or concomitantly with treatment (angioplasty), as described above, of the ischemic heart disease and a second administration of the two administrations is within 96 hours from the diagnosis and/or angioplasty.

According to a specific embodiment, the first administration of the at least 2 administrations is immediately after diagnosis e.g., up to 12 hours following diagnosis of the ischemic heart disease and a second administration of the two administrations is within 48-96 hours from said diagnosis.

According to a specific embodiment, the therapeutically effective amount is 20-50 μg/Kg.

According to a specific embodiment, the therapeutically effective amount is 25-50 μg/Kg.

According to a specific embodiment, the therapeutically effective amount is 30-50 μg/Kg.

According to a specific embodiment, the therapeutically effective amount is 30-40 μg/Kg.

According to a specific embodiment, the therapeutically effective amount is 30-35 μg/Kg.

According to a specific embodiment, the therapeutically effective amount is 20-40 μg/Kg.

According to a specific embodiment, the therapeutically effective amount is 20-45 μg/Kg.

According to a specific embodiment, the therapeutically effective amount is 30-45 μg/Kg.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

Agrin treatment as described herein can be combined with other treatment modalities. These other treatments include medication (e.g., blood pressure medication, calcium channel blockers, digitalis, anti-arrhythmics, ACE inhibitors, anti-coagulants, immunosuppressants, pain relievers, vasodilators, etc.), angioplasty, stent placement, coronary artery bypass graft, cardiac assist device (e.g., left ventricular assist device, balloon pump), pacemaker placement, heart transplantation, etc. In certain embodiments, the agent provides a bridge to recover for a subject waiting to undergo heart transplantation.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, CA (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

Animals

Pigs were purchased from Veterinary Medicine, LMU Munich (Oberschleil3heim, Germany). Animal care and all experimental procedures were performed in strict accordance to the German and National Institutes of Health animal legislation guidelines and were approved by the Bavarian Animal Care and Use Committee.

For the rat experiments, Sprague Dawley (SD) rats at 8 weeks old Sprague Dawley (SD) rats were used. All experiments were approved by the Animal Care and Use Committee of the Weizmann Institute of Science.

Pig Ischemia/Reperfusion Model

All pig experiments were conducted at the Walter-Brendel Center for Experimental Medicine at the University of Munich. Pigs (n=3 per Saline grooup, n=3 per single Agrin treatment group and n=4 for double Agrin treatment group) were instrumented as previously described¹¹. Briefly, a percutaneous transluminal coronary angioplasty balloon was placed in the left anterior descending artery distal to the first diagonal branch and inflated with 10 atm for 60 minutes. Correct localization of the coronary occlusion and patency of the first diagonal branch were ensured by injection of contrast agent under fluoroscopy. In all groups, the percutaneous transluminal coronary angioplasty balloon was partly or completely deflated after 60 minutes of ischemia; the onset of reperfusion was documented angiographically.

Agrin Administration

Human recombinant Agrin (rAgrin, 6624-AG, R&D biosystems, USA) was used at a concentration of 0.327 mg/mL in PBS. Animals in all treatment groups were administered with 33 mg/Kg. Anterograde treatment was performed by partly deflating the angioplasty balloon to 6 atm, and injecting the Agrin “over the wire” at a duration of 3′. Retrograde treatment was one by introducing a catheter into the great cardiac vein and injection at a 30 ml/hr pace, 5′ before and 5′ after reperfusion. Intramyocardial injection was performed using C-cath catheter (Celyad, Belgium), according to manufacturer instructions. Sterile saline was used as control.

Agrin Distribution Analysis

For Agrin distribution analysis, hearts were harvested after 60′ of reperfusion. Heart were dissected into 36 sections, as previously described¹¹. Protein was extracted from each section using mechanical disassociation (Rotor-stator homoginizer) into RIPA. As rAgrin carries a N′-Histag, it was immunopercipitated using Ni-NTA agarose beads (#30210, Qiagen, USA). IP products were subjected to Western blot analysis. Protein intensity for each WB were acquired, normalized to the positive control (5 ng of rAgrin) and the average intensity of each section in each group was determined.

Heart Function Analysis

Heart ejection function (EF) was determined using fluoroscopy image analysis. EF was acquired before ischemia after 3 and 28 days of reperfusion.

Hemodynamic Measurements

Left ventricular end-diastolic pressure and ejection fraction measurements were performed before ischemia and after 3 and 28 days of reperfusion, using a standard pressure-volume loop (PV-loop) percutaneously introduced (pig tail catheter) into the left ventricle lumen (ADV500, Transonic, USA).

Infarct Size

Infarct size was assessed via methylene blue exclusion, tetrazolium red viability staining as described previouslyl¹¹. Alternatively, infarct size was estimated after 25 days of reperfusion using late enhancement reaction of contrast agent under MRI scan.

Cardiomyocyte Size

Hearts were harvested after 28 days of reperfusion, and sectioned as above described. Representative sections of the border zone were stained with Wheat Germ Agglutinin (WGA) to image the CM membranes. Image analysis was performed using ImageJ software.

Subendocardial Segment Shortening (SES)

At the end of the experiment (28 days post MI), sternotomy was performed, and ultrasonic crystals were placed in the control, border zone and infarcted areas. Subendocardial segment shortening (SES) was performed under resting heart rate as well as at 120/min and 150/min atrial pacing (for 1 minute each).

Rat Delayed Treatment (CHF) Model

For the Rat CHF model, we have performed a left artery descending (LAD) permanent ligation, as described in (Bassat et al., 2017). Briefly, 8 weeks old SD rats were sedated with isoflurane (Abbott Laboratories) and, following tracheal intubation, were artificially ventilated. Following skin incision, lateral thoracotomy at the third intercostal space was performed by blunt dissection of the intercostal muscles. Following artery ligation, intramyocardial injections of Agrin (0.33 μg/Kg in 200 ul of Saline) or Saline were administered. Then, thoracic wall incisions were sutured using surgical grade adhesive (Histoacril, B.Braun, Germany). Heart function was assed using ultrasound echocardiography (Vevo3100, Visualsonics).

Statistical Analysis

The results are given as mean±SEM. Statistical analysis were performed by 1-way ANOVA. Whenever a significant effect was obtained with ANOVA, multiple-comparison tests between the groups with the Student-Newman-Keuls procedure were performed (SPSS 20.0 statistical program or Graphpad).

### EXAMPLE 1

### Local Delivery Method of Agrin into the Ischemic Regions of the Heart

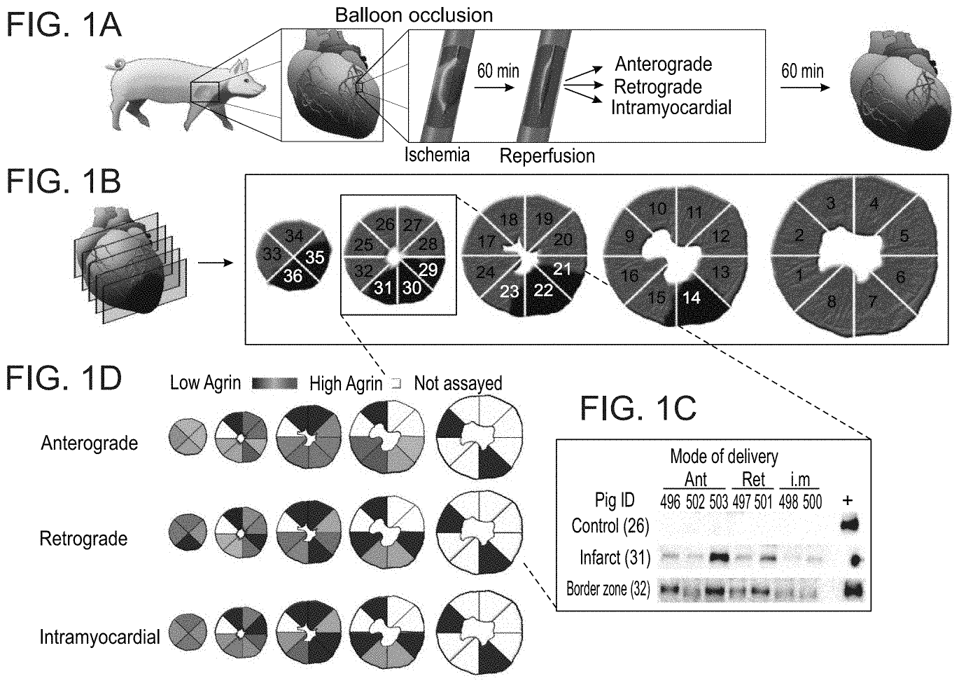

In search for an efficient way to specifically deliver Agrin into the infarcted heart, the ischemia-reperfusion model of MI in pigs was used, as depicted in FIG. 1A. The LAD was occluded for an hour, reperfused and treated with 33 μg/Kg human rAgrin (C′ Agrin, 6624-AG, R&D systems) at reperfusion, delivered in one of three methods: anterograde (autoperfusion into the LAD), retrograde (into the great cardiac vein)⁶, or intramyocardial (into the left ventricle wall, through the endocardium). Anterograde injection was performed using the same catheter used for reperfusion, while retrograde injection included catheterization of the great cardiac vein (directed through the right Jugular vein). Intramyocardial injection was performed using the C-cath catheter (Celyad), injecting through the endocardium at 9 different hypokinetic region of the left ventricular wall. After an additional 60 minutes, hearts were harvested, sectioned into 36 segments, and each segment was annotated as infarct (injury), border zone or control (unaffected) (FIG. 1B). To examine Agrin distribution, the protein was extracted using mechanical means into denaturative RIPA buffer from the different segments. Agrin was immunoprecipitated using Ni-NTA beads from each extract sample and compared by Western blotting with anti-His tag, a moiety attached to the c-terminal part of Agrin (demonstrated in FIG. 1C). Averaging the different Agrin distribution in every sample in the 7 analyzed pigs, revealed that the anterograde trajectory showed the most intense Agrin retention in the treated hearts (FIG. 1D). Interestingly, the levels of Agrin were higher in the infarct and border zone sections and almost none at remote regions of the heart (FIG. 1D), suggesting a specific short-distance targeting of Agrin to clinically relevant target sites. Collectively, these data highlight the anterograde trajectory as an efficient and specific method that can be utilized for Agrin delivery to the relevant parts of the infarcted heart.

### EXAMPLE 2

### Agrin Improves Heart Function in Pigs after Acute MI

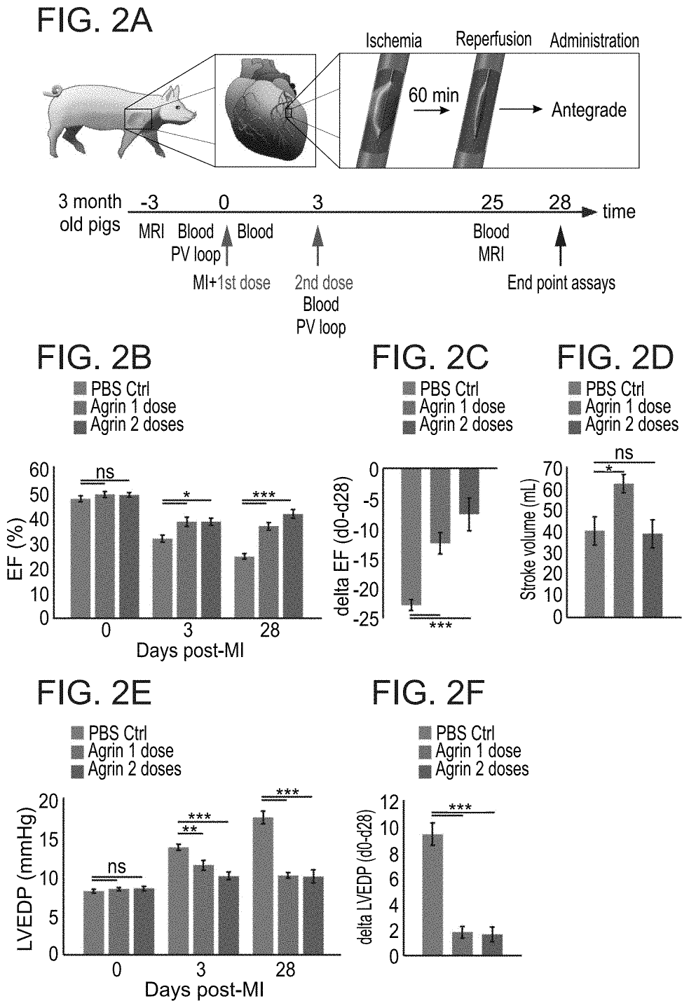

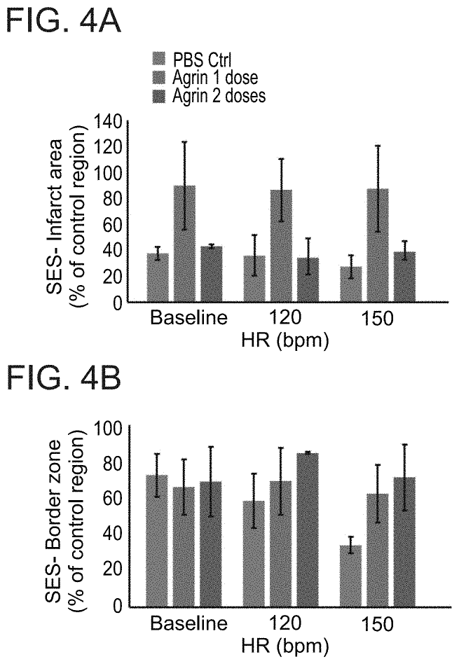

The effectiveness of Agrin treatment after MI in pigs was tested. To do so, infarcted pigs were treated with rhAg (at 33 μg/Kg) or saline (control) either immediately after reperfusion, or adding a second dose 3 days post MI using the antegrade method (FIG. 2A). As shown, rhAg treated hearts had improved heart function. Post ischemic loss of the ejection fraction (EF) parameter, measured by fluoroscopy, was reduced after rhAg administration (FIGS. 2B-C). Second, the stroke volume, i.e., the amount of blood pumped into the aorta during each stroke, increased in the rhAg (single dose only) treated animals, as measured by MRI (FIG. 2D). Another important parameter for heart function is left ventricular end-diastolic pressure (LVEDP), which increases post MI due to the fact that injured hearts display increased rigidity. Interestingly, rhAg treatment prevented, almost completely, the MI-induced LVEDP increase (FIGS. 2E-F). An increase in the Subendocardial segment shortening (SES) of the infarct region in rhAg treated hearts was observed (FIGS. 4A-B) and to some extent also in the border zone (see 150 bpm, FIG. 4B). The fact that the 2 doses Agrin treated animals did not show that SES increase is maybe due to the small sample size, as only 2 animals could be recorded in this group. This SES increase indicates that Agrin treatment improves the myocardium contraction of the infarct region, improving heart function. Both EF (FIG. 2C) and LVEDP (FIG. 2E) parameters showed short-term rapid improvement already 3 days post MI. Taken together, these findings suggest that Agrin can improve cardiac function post MI, and show indications for a cardio protective mechanism of action.

### EXAMPLE 3

### Agrin Reduces Scar Tissue and Prevents Remodeling of the Infarcted Heart

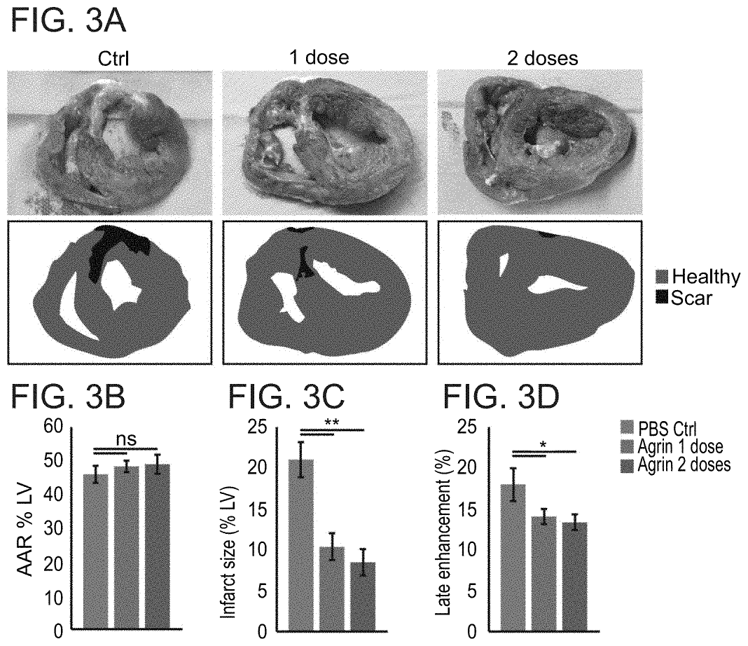

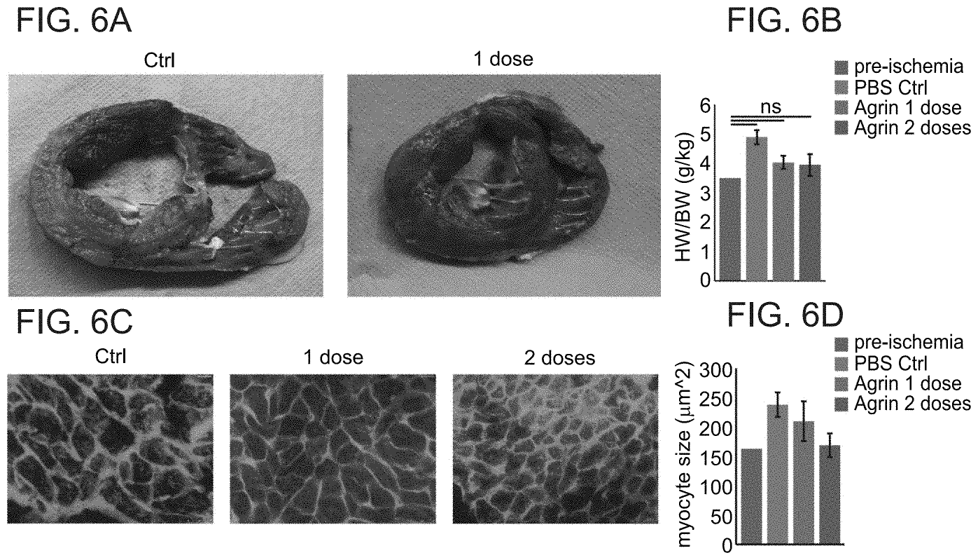

One of the most prominent and harmful consequences of MI is the excessive scar expansion and adverse remodeling of the heart, which includes CM hypertrophy, ventricular dilation and overall increased heart weight. Therefore, we examined these parameters in the infarcted pigs' hearts. Scarring within the infarcted hearts was reduced significantly in the rhAg treated animals (FIGS. 3A-D), as assessed by both triphenyltetrazolium chloride (TTC) staining (FIG. 3C) and MRI (Late enhancement, FIG. 3D). The increase in heart weight observed after MI is another prevalent remodeling feature of injured animals, which was reduced in rhAg treated hearts 28 days post MI, presented as heart to body weight ratio (HW/BW) (FIG. 6A). In fact, rhAg treated animals resembled the pre-ischemic parameter (FIG. 6B). Finally, heart remodeling is associated with CM hypertrophy, which naturally happens after injury to augment ventricle wall force due to the loss of myocardium. In the rhAg treated hearts (only in the 2 doses) we observed preliminary indications for reduction in CM size, compared to saline control (FIGS. 6C-D). Taken together, our data collectively suggest that Agrin prevents cardiac scarring and remodeling post MI in pigs.

### EXAMPLE 4

### Agrin Improves Heart Function in a Rodent Model for CHF

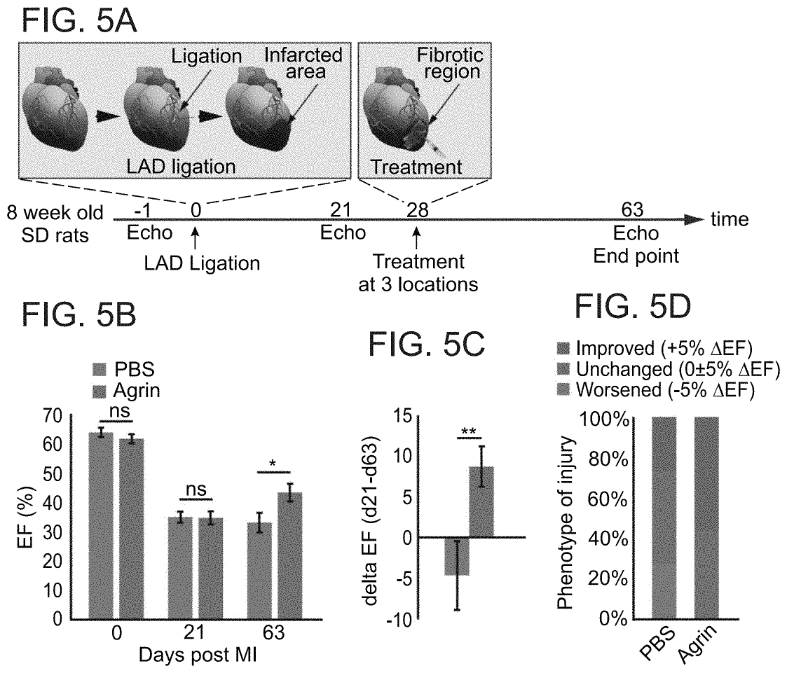

The severity of the clinical symptoms associated with CHF, the ability of Agrin to alleviate these symptoms was tested. A rodent model of CHF, mimicking heart failure caused by post MI deterioration, was established. Specifically, MI was induced by permanent LAD ligation in rats, and the animals were allowed to recover for 28 days, and then injected either rat recombinant Agrin (rrAg, #550-AG-100, R&D systems, USA), or saline intramyocardial (i.e. as done in mice, (Bassat et al., 2017 Nature 547, 179-184) (FIG. 5A). The 28 days' time point was chosen as this is considered to be a phase in which the resulting scar is mature, and there is little to no immune response (Liehn et al., 2011 J Am Coll Cardiol 58, 2357-2362; Willems et al., 1994 Am J Pathol 145, 868-875). Interestingly, it was found that heart function, as measured by EF, was significantly improved in the rrAg treated rats (FIGS. 5B-C). Further, when the distribution of animals according to their EF difference between treatment and endpoint was examined, it was found that the fraction of animals showing heart function improvement was increased in the rrAg treated animals (FIG. 5D). Moreover, while most of the Saline treated animals showed either deteriorated heart function or did not change, none of the rrAg treated animals showed decreased EF. Taken together, these findings suggest the possibility that Agrin can have beneficial effects on the function of failed hearts, and as such a new therapeutic agent for CHF patients.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

### Other References are included in the Document

1. Organization, W. h. World Health Statistics 2016. (2016).
2. Sorita, A. et al. Off-hour presentation and outcomes in patients with acute myocardial infarction: systematic review and meta-analysis. BMJ 348, f7393, doi:10.1136/bmj.f7393 (2014).
3. Toldo, S. & Abbate, A. The NLRP3 inflammasome in acute myocardial infarction. Nat Rev Cardiol, doi:10.1038/nrcardio.2017.161 (2017).
4. Tzahor, E. & Poss, K. D. Cardiac regeneration strategies: Staying young at heart. Science 356, 1035-1039, doi:10.1126/science.aam5894 (2017).
5. Bassat, E. et al. The extracellular matrix protein agrin promotes heart regeneration in mice. Nature 547, 179-184, doi:10.1038/nature22978 (2017).
6. Hinkel, R., Trenkwalder, T. & Kupatt, C. Gene therapy for ischemic heart disease. Expert Opin. Biol Ther 11, 723-737, doi:10.1517/14712598.2011.570749 [doi] (2011).
7. Chong, J. J. et al. Human embryonic-stem-cell-derived cardiomyocytes regenerate non-human primate hearts. Nature, doi:nature13233 [pii];10.1038/nature13233 [doi] (2014).
8. Battler, A. et al. Intracoronary injection of basic fibroblast growth factor enhances angiogenesis in infarcted swine myocardium. J Am Coll. Cardiol 22, 2001-2006 (1993).
9. Wei, K. et al. Epicardial FSTL1 reconstitution regenerates the adult mammalian heart. Nature 525, 479-485 (2015).
10. Bersell, K., Arab, S., Haring, B. & Kuhn, B. Neuregulinl/ErbB4 Signaling Induces Cardiomyocyte Proliferation and Repair of Heart Injury. Cell 138, 257-270, doi(dot)www(dot)doi(dot)org/10.1016/j(dot)cell(dot)2009(dot)04(dot)060 (2009).
11. Hinkel, R. et al. Inhibition of MicroRNA-92a Protects Against Ischemia-Reperfusion Injury in a Large Animal Model. Circulation 128, 1066-1075 (2013).
12. Jessup, M. et al. Calcium Upregulation by Percutaneous Administration of Gene Therapy in Cardiac Disease (CUPID): a phase 2 trial of intracoronary gene therapy of sarcoplasmic reticulum Ca2+-ATPase in patients with advanced heart failure. Circulation 124, 304-313, doi:CIRCULATIONAHA.111.022889 [pii];10.1161/CIRCULATIONAHA.111.022889 [doi] (2011).
13. Schachinger, V. et al. Intracoronary bone marrow-derived progenitor cells in acute myocardial infarction. N Engl J Med 355, 1210-1221 (2006).
14. Hinkel, R. et al. Heme Oxygenase-1 Gene Therapy Provides Cardioprotection Via Control of Post-Ischemic Inflammation: An Experimental Study in a Pre-Clinical Pig Model. Journal of the American College of Cardiology 66, 154-165 (2015).
15. Rossello, X. & Yellon, D. M. The RISK pathway and beyond. Basic Research in Cardiology 113, 2, doi:10.1007/s00395-017-0662-x (2017).

What is claimed is:

1. A method of treating an ischemic heart disease in a subject in need thereof, the method comprising administering to the subject a therapeutically effective amount of Agrin in an anterograde intracoronary manner, thereby treating the ischemic heart disease in the subject.

2. The method of claim 1, wherein said ischemic heart disease is selected from the group consisting of acute myocardial infarction (AMI), myocardial infarction (MI) and Chronic heart failure (CHF).

3. The method of claim 1, wherein said therapeutically effective amount comprises a single administration.

4. The method of claim 1, wherein said therapeutically effective amount comprises a repeated administration.

5. The method of claim 4, wherein said repeated administration comprises at least 2 administrations.

6. The method of claim 5, wherein a first administration of said at least 2 administrations is immediately after diagnosis of the ischemic heart disease and a second administration of said two administrations is within 96 hours from said diagnosis.

7. The method of claim 6, wherein a first administration of said at least 2 administrations is immediately after diagnosis of the ischemic heart disease and a second administration of said two administrations is within 48-96 hours from said diagnosis.

8. The method of claim 1, wherein said therapeutically effective amount is 20-50 μg/Kg.

9. The method of claim 1, wherein said Agrin is an Agrin peptide capable of inducing cardiomyocyte proliferation.

10. The method of claim 1, wherein said Agrin is not a part of a fusion polypeptide.

11. The method of claim 1, wherein said Agrin is in a soluble form.

12. The method of claim 1, wherein said Agrin peptide comprises a laminin G-like 1 domain (G1) and a laminin G-like 2 domain (G2).

13. The method of claim 1, wherein said Agrin peptide comprises a laminin G-like 1 domain (G1) and a laminin G-like 2 domain (G2) and is devoid of a laminin G-like 3 domain (G3).

14. The method of claim 1, wherein said Agrin peptide is 150-600 amino acids long.

15. The method of claim 1, wherein said Agrin peptide is 200-600 amino acids long.

16. The method of claim 1 , wherein said Agrin peptide is 200-520 amino acids long.

17. The method of claim 1, wherein said Agrin peptide is 400-520 amino acids long.

18. The method of claim 1, wherein said Agrin peptide is 300-520 amino acids long.

19. The method of claim 1, wherein said Agrin peptide is human agrin.

20. The method of claim 1, wherein said Agrin comprises a fragment of human Agrin.


SEQUENCE LISTING (38 sequences)

SEQ ID NO: 1 - protein, 4392 aa, Homo sapiens
MGWRAAGALLLALLLHGRLLAVTHGLRAYDGLSLPEDIETVTASQMRWTHSYLSDDEDMLADSISGDDLGSGDLGSGDFQMVYFRALVNFTRSIEYSPQLEDAGSREFREVSEAVVDTLESEYLKIPGDQVVSVVFIKELDGWVFVELDVGSEGNADGAQIQEMLLRVISSGSVASYVTSPQGFQFRRLGTVPQFPRACTEAEFACHSYNECVALEYRCDRRPDCRDMSDELNCEEPVLGISPTFSLLVETTSLPPRPETTIMRQPPVTHAPQPLLPGSVRPLPCGPQEAACRNGHCIPRDYLCDGQEDCEDGSDELDCGPPPPCEPNEFPCGNGHCALKLWRCDGDFDCEDRTDEANCPTKRPEEVCGPTQFRCVSTNMCIPASFHCDEESDCPDRSDEFGCMPPQVVTPPRESIQASRGQTVTFTCVAIGVPTPIINWRLNWGHIPSHPRVTVTSEGGRGTLIIRDVKESDQGAYTCEAMNARGMVFGIPDGVLELVPQRAGPCPDGHFYLEHSAACLPCFCFGITSVCQSTRRFRDQIRLRFDQPDDFKGVNVTMPAQPGTPPLSSTQLQIDPSLHEFQLVDLSRRFLVHDSFWALPEQFLGNKVDSYGGSLRYNVRYELARGMLEPVQRPDVVLMGAGYRLLSRGHTPTQPGALNQRQVQFSEEHWVHESGRPVQRAELLQVLQSLEAVLIQTVYNTKMASVGLSDIAMDTTVTHATSHGRAHSVEECRCPIGYSGLSCESCDAHFTRVPGGPYLGTCSGCNCNGHASSCDPVYGHCLNCQHNTEGPQCNKCKAGFFGDAMKATATSCRPCPCPYIDASRRFSDTCFLDTDGQATCDACAPGYTGRRCESCAPGYEGNPIQPGGKCRPVNQEIVRCDERGSMGTSGEACRCKNNVVGRLCNECADGSFHLSTRNPDGCLKCFCMGVSRHCTSSSWSRAQLHGASEEPGHFSLTNAASTHTTNEGIFSPTPGELGFSSFHRLLSGPYFWSLPSRFLGDKVTSYGGELRFTVTQRSQPGSTPLHGQPLVVLQGNNIILEHHVAQEPSPGQPSTFIVPFREQAWQRPDGQPATREHLLMALAGIDTLLIRASYAQQPAESRVSGISMDVAVPEETGQDPALEVEQCSCPPGYRGPSCQDCDTGYTRTPSGLYLGTCERCSCHGHSEACEPETGACQGCQHHTEGPRCEQCQPGYYGDAQRGTPQDCQLCPCYGDPAAGQAAHTCFLDTDGHPTCDACSPGHSGRHCERCAPGYYGNPSQGQPCQRDSQVPGPIGCNCDPQGSVSSQCDAAGQCQCKAQVEGLTCSHCRPHHFHLSASNPDGCLPCFCMGITQQCASSAYTRHLISTHFAPGDFQGFALVNPQRNSRLTGEFTVEPVPEGAQLSFGNFAQLGHESFYWQLPETYQGDKVAAYGGKLRYTLSYTAGPQGSPLSDPDVQITGNNIMLVASQPALQGPERRSYEIMFREEFWRRPDGQPATREHLLMALADLDELLIRATFSSVPLAASISAVSLEVAQPGPSNRPRALEVEECRCPPGYIGLSCQDCAPGYTRTGSGLYLGHCELCECNGHSDLCHPETGACSQCQHNAAGEFCELCAPGYYGDATAGTPEDCQPCACPLTNPENMFSRTCESLGAGGYRCTACEPGYTGQYCEQCGPGYVGNPSVQGGQCLPETNQAPLVVEVHPARSIVPQGGSHSLRCQVSGSPPHYFYWSREDGRPVPSGTQQRHQGSELHFPSVQPSDAGVYICTCRNLHQSNTSRAELLVTEAPSKPITVTVEEQRSQSVRPGADVTFICTAKSKSPAYTLVWTRLHNGKLPTRAMDFNGILTIRNVQLSDAGTYVCTGSNMFAMDQGTATLHVQASGTLSAPVVSIHPPQLTVQPGQLAEFRCSATGSPTPTLEWTGGPGGQLPAKAQIHGGILRLPAVEPTDQAQYLCRAHSSAGQQVARAVLHVHGGGGPRVQVSPERTQVHAGRTVRLYCRAAGVPSATITWRKEGGSLPPQARSERTDIATLLIPAITTADAGFYLCVATSPAGTAQARIQVVVLSASDASPPPVKIESSSPSVTEGQTLDLNCVVAGSAHAQVTWYRRGGSLPPHTQVHGSRLRLPQVSPADSGEYVCRVENGSGPKEASITVSVLHGTHSGPSYTPVPGSTRPIRIEPSSSHVAEGQTLDLNCVVPGQAHAQVTWHKRGGSLPARHQTHGSLLRLHQVTPADSGEYVCHVVGTSGPLEASVLVTIEASVIPGPIPPVRIESSSSTVAEGQTLDLSCVVAGQAHAQVTWYKRGGSLPARHQVRGSRLYIFQASPADAGQYVCRASNGMEASITVTVTGTQGANLAYPAGSTQPIRIEPSSSQVAEGQTLDLNCVVPGQSHAQVTWHKRGGSLPVRHQTHGSLLRLYQASPADSGEYVCRVLGSSVPLEASVLVTIEPAGSVPALGVTPTVRIESSSSQVAEGQTLDLNCLVAGQAHAQVTWHKRGGSLPARHQVHGSRLRLLQVTPADSGEYVCRVVGSSGTQEASVLVTIQQRLSGSHSQGVAYPVRIESSSASLANGHTLDLNCLVASQAPHTITWYKRGGSLPSRHQIVGSRLRIPQVTPADSGEYVCHVSNGAGSRETSLIVTIQGSGSSHVPSVSPPIRIESSSPTVVEGQTLDLNCVVARQPQAIITWYKRGGSLPSRHQTHGSHLRLHQMSVADSGEYVCRANNNIDALEASIVISVSPSAGSPSAPGSSMPIRIESSSSHVAEGETLDLNCVVPGQAHAQVTWHKRGGSLPSHHQTRGSRLRLHHVSPADSGEYVCRVMGSSGPLEASVLVTIEASGSSAVHVPAPGGAPPIRIEPSSSRVAEGQTLDLKCVVPGQAHAQVTWHKRGGNLPARHQVHGPLLRLNQVSPADSGEYSCQVTGSSGTLEASVLVTIEPSSPGPIPAPGLAQPIYIEASSSHVTEGQTLDLNCVVPGQAHAQVTWYKRGGSLPARHQTHGSQLRLHLVSPADSGEYVCRAASGPGPEQEASFTVTVPPSEGSSYRLRSPVISIDPPSSTVQQGQDASFKCLIHDGAAPISLEWKTRNQELEDNVHISPNGSIITIVGTRPSNHGTYRCVASNAYGVAQSVVNLSVHGPPTVSVLPEGPVWVKVGKAVTLECVSAGEPRSSARWTRISSTPAKLEQRTYGLMDSHAVLQISSAKPSDAGTYVCLAQNALGTAQKQVEVIVDTGAMAPGAPQVQAEEAELTVEAGHTATLRCSATGSPAPTIHWSKLRSPLPWQHRLEGDTLIIPRVAQQDSGQYICNATSPAGHAEATIILHVESPPYATTVPEHASVQAGETVQLQCLAHGTPPLTFQWSRVGSSLPGRATARNELLHFERAAPEDSGRYRCRVTNKVGSAEAFAQLLVQGPPGSLPATSIPAGSTPTVQVTPQLETKSIGASVEFHCAVPSDRGTQLRWFKEGGQLPPGHSVQDGVLRIQNLDQSCQGTYICQAHGPWGKAQASAQLVIQALPSVLINIRTSVQTVVVGHAVEFECLALGDPKPQVTWSKVGGHLRPGIVQSGGVVRIAHVELADAGQYRCTATNAAGTTQSHVLLLVQALPQISMPQEVRVPAGSAAVFPCIASGYPTPDISWSKLDGSLPPDSRLENNMLMLPSVRPQDAGTYVCTATNRQGKVKAFAHLQVPERVVPYFTQTPYSFLPLPTIKDAYRKFEIKITFRPDSADGMLLYNGQKRVPGSPTNLANRQPDFISFGLVGGRPEFRFDAGSGMATIRHPTPLALGHFHTVTLLRSLTQGSLIVGDLAPVNGTSQGKFQGLDLNEELYLGGYPDYGAIPKAGLSSGFIGCVRELRIQGEEIVFHDLNLTAHGISHCPTCRDRPCQNGGQCHDSESSSYVCVCPAGFTGSRCEHSQALHCHPEACGPDATCVNRPDGRGYTCRCHLGRSGLRCEEGVTVTTPSLSGAGSYLALPALTNTHHELRLDVEFKPLAPDGVLLFSGGKSGPVEDFVSLAMVGGHLEFRYELGSGLAVLRSAEPLALGRWHRVSAERLNKDGSLRVNGGRPVLRSSPGKSQGLNLHTLLYLGGVEPSVPLSPATNMSAHFRGCVGEVSVNGKRLDLTYSFLGSQGIGQCYDSSPCERQPCQHGATCMPAGEYEFQCLCRDGFKGDLCEHEENPCQLREPCLHGGTCQGTRCLCLPGFSGPRCQQGSGHGIAESDWHLEGSGGNDAPGQYGAYFHDDGFLAFPGHVFSRSLPEVPETIELEVRTSTASGLLLWQGVEVGEAGQGKDFISLGLQDGHLVFRYQLGSGEARLVSEDPINDGEWHRVTALREGRRGSIQVDGEELVSGRSPGPNVAVNAKGSVYIGGAPDVATLTGGRFSSGITGCVKNLVLHSARPGAPPPQPLDLQHRAQAGANTRPCPS

SEQ ID NO: 2 - protein, 4391 aa, Homo sapiens
MGWRAAGALLLALLLHGRLLAVTHGLRAYDGLSLPEDIETVTASQMRWTHSYLSDDEDMLADSISGDDLGSGDLGSGDFQMVYFRALVNFTRSIEYSPQLEDAGSREFREVSEAVVDTLESEYLKIPGDQVVSVVFIKELDGWVFVELDVGSEGNADGAQIQEMLLRVISSGSVASYVTSPQGFQFRRLGTVPQFPRACTEAEFACHSYNECVALEYRCDRRPDCRDMSDELNCEEPVLGISPTFSLLVETTSLPPRPETTIMRQPPVTHAPQPLLPGSVRPLPCGPQEAACRNGHCIPRDYLCDGQEDCEDGSDELDCGPPPPCEPNEFPCGNGHCALKLWRCDGDFDCEDRTDEANCPTKRPEEVCGPTQFRCVSTNMCIPASFHCDEESDCPDRSDEFGCMPPQVVTPPRESIQASRGQTVTFTCVAIGVPTPIINWRLNWGHIPSHPRVTVTSEGGRGTLIIRDVKESDQGAYTCEAMNARGMVFGIPDGVLELVPQRGPCPDGHFYLEHSAACLPCFCFGITSVCQSTRRFRDQIRLRFDQPDDFKGVNVTMPAQPGTPPLSSTQLQIDPSLHEFQLVDLSRRFLVHDSFWALPEQFLGNKVDSYGGSLRYNVRYELARGMLEPVQRPDVVLMGAGYRLLSRGHTPTQPGALNQRQVQFSEEHWVHESGRPVQRAELLQVLQSLEAVLIQTVYNTKMASVGLSDIAMDTTVTHATSHGRAHSVEECRCPIGYSGLSCESCDAHFTRVPGGPYLGTCSGCNCNGHASSCDPVYGHCLNCQHNTEGPQCNKCKAGFFGDAMKATATSCRPCPCPYIDASRRFSDTCFLDTDGQATCDACAPGYTGRRCESCAPGYEGNPIQPGGKCRPVNQEIVRCDERGSMGTSGEACRCKNNVVGRLCNECADGSFHLSTRNPDGCLKCFCMGVSRHCTSSSWSRAQLHGASEEPGHFSLTNAASTHTTNEGIFSPTPGELGFSSFHRLLSGPYFWSLPSRFLGDKVTSYGGELRFTVTQRSQPGSTPLHGQPLVVLQGNNIILEHHVAQEPSPGQPSTFIVPFREQAWQRPDGQPATREHLLMALAGIDTLLIRASYAQQPAESRVSGISMDVAVPEETGQDPALEVEQCSCPPGYRGPSCQDCDTGYTRTPSGLYLGTCERCSCHGHSEACEPETGACQGCQHHTEGPRCEQCQPGYYGDAQRGTPQDCQLCPCYGDPAAGQAAHTCFLDTDGHPTCDACSPGHSGRHCERCAPGYYGNPSQGQPCQRDSQVPGPIGCNCDPQGSVSSQCDAAGQCQCKAQVEGLTCSHCRPHHFHLSASNPDGCLPCFCMGITQQCASSAYTRHLISTHFAPGDFQGFALVNPQRNSRLTGEFTVEPVPEGAQLSFGNFAQLGHESFYWQLPETYQGDKVAAYGGKLRYTLSYTAGPQGSPLSDPDVQITGNNIMLVASQPALQGPERRSYEIMFREEFWRRPDGQPATREHLLMALADLDELLIRATFSSVPLAASISAVSLEVAQPGPSNRPRALEVEECRCPPGYIGLSCQDCAPGYTRTGSGLYLGHCELCECNGHSDLCHPETGACSQCQHNAAGEFCELCAPGYYGDATAGTPEDCQPCACPLTNPENMFSRTCESLGAGGYRCTACEPGYTGQYCEQCGPGYVGNPSVQGGQCLPETNQAPLVVEVHPARSIVPQGGSHSLRCQVSGSPPHYFYWSREDGRPVPSGTQQRHQGSELHFPSVQPSDAGVYICTCRNLHQSNTSRAELLVTEAPSKPITVTVEEQRSQSVRPGADVTFICTAKSKSPAYTLVWTRLHNGKLPTRAMDFNGILTIRNVQLSDAGTYVCTGSNMFAMDQGTATLHVQASGTLSAPVVSIHPPQLTVQPGQLAEFRCSATGSPTPTLEWTGGPGGQLPAKAQIHGGILRLPAVEPTDQAQYLCRAHSSAGQQVARAVLHVHGGGGPRVQVSPERTQVHAGRTVRLYCRAAGVPSATITWRKEGGSLPPQARSERTDIATLLIPAITTADAGFYLCVATSPAGTAQARIQVVVLSASDASPPPVKIESSSPSVTEGQTLDLNCVVAGSAHAQVTWYRRGGSLPPHTQVHGSRLRLPQVSPADSGEYVCRVENGSGPKEASITVSVLHGTHSGPSYTPVPGSTRPIRIEPSSSHVAEGQTLDLNCVVPGQAHAQVTWHKRGGSLPARHQTHGSLLRLHQVTPADSGEYVCHVVGTSGPLEASVLVTIEASVIPGPIPPVRIESSSSTVAEGQTLDLSCVVAGQAHAQVTWYKRGGSLPARHQVRGSRLYIFQASPADAGQYVCRASNGMEASITVTVTGTQGANLAYPAGSTQPIRIEPSSSQVAEGQTLDLNCVVPGQSHAQVTWHKRGGSLPVRHQTHGSLLRLYQASPADSGEYVCRVLGSSVPLEASVLVTIEPAGSVPALGVTPTVRIESSSSQVAEGQTLDLNCLVAGQAHAQVTWHKRGGSLPARHQVHGSRLRLLQVTPADSGEYVCRVVGSSGTQEASVLVTIQQRLSGSHSQGVAYPVRIESSSASLANGHTLDLNCLVASQAPHTITWYKRGGSLPSRHQIVGSRLRIPQVTPADSGEYVCHVSNGAGSRETSLIVTIQGSGSSHVPSVSPPIRIESSSPTVVEGQTLDLNCVVARQPQAIITWYKRGGSLPSRHQTHGSHLRLHQMSVADSGEYVCRANNNIDALEASIVISVSPSAGSPSAPGSSMPIRIESSSSHVAEGETLDLNCVVPGQAHAQVTWHKRGGSLPSHHQTRGSRLRLHHVSPADSGEYVCRVMGSSGPLEASVLVTIEASGSSAVHVPAPGGAPPIRIEPSSSRVAEGQTLDLKCVVPGQAHAQVTWHKRGGNLPARHQVHGPLLRLNQVSPADSGEYSCQVTGSSGTLEASVLVTIEPSSPGPIPAPGLAQPIYIEASSSHVTEGQTLDLNCVVPGQAHAQVTWYKRGGSLPARHQTHGSQLRLHLVSPADSGEYVCRAASGPGPEQEASFTVTVPPSEGSSYRLRSPVISIDPPSSTVQQGQDASFKCLIHDGAAPISLEWKTRNQELEDNVHISPNGSIITIVGTRPSNHGTYRCVASNAYGVAQSVVNLSVHGPPTVSVLPEGPVWVKVGKAVTLECVSAGEPRSSARWTRISSTPAKLEQRTYGLMDSHAVLQISSAKPSDAGTYVCLAQNALGTAQKQVEVIVDTGAMAPGAPQVQAEEAELTVEAGHTATLRCSATGSPAPTIHWSKLRSPLPWQHRLEGDTLIIPRVAQQDSGQYICNATSPAGHAEATIILHVESPPYATTVPEHASVQAGETVQLQCLAHGTPPLTFQWSRVGSSLPGRATARNELLHFERAAPEDSGRYRCRVTNKVGSAEAFAQLLVQGPPGSLPATSIPAGSTPTVQVTPQLETKSIGASVEFHCAVPSDRGTQLRWFKEGGQLPPGHSVQDGVLRIQNLDQSCQGTYICQAHGPWGKAQASAQLVIQALPSVLINIRTSVQTVVVGHAVEFECLALGDPKPQVTWSKVGGHLRPGIVQSGGVVRIAHVELADAGQYRCTATNAAGTTQSHVLLLVQALPQISMPQEVRVPAGSAAVFPCIASGYPTPDISWSKLDGSLPPDSRLENNMLMLPSVRPQDAGTYVCTATNRQGKVKAFAHLQVPERVVPYFTQTPYSFLPLPTIKDAYRKFEIKITFRPDSADGMLLYNGQKRVPGSPTNLANRQPDFISFGLVGGRPEFRFDAGSGMATIRHPTPLALGHFHTVTLLRSLTQGSLIVGDLAPVNGTSQGKFQGLDLNEELYLGGYPDYGAIPKAGLSSGFIGCVRELRIQGEEIVFHDLNLTAHGISHCPTCRDRPCQNGGQCHDSESSSYVCVCPAGFTGSRCEHSQALHCHPEACGPDATCVNRPDGRGYTCRCHLGRSGLRCEEGVTVTTPSLSGAGSYLALPALTNTHHELRLDVEFKPLAPDGVLLFSGGKSGPVEDFVSLAMVGGHLEFRYELGSGLAVLRSAEPLALGRWHRVSAERLNKDGSLRVNGGRPVLRSSPGKSQGLNLHTLLYLGGVEPSVPLSPATNMSAHFRGCVGEVSVNGKRLDLTYSFLGSQGIGQCYDSSPCERQPCQHGATCMPAGEYEFQCLCRDGFKGDLCEHEENPCQLREPCLHGGTCQGTRCLCLPGFSGPRCQQGSGHGIAESDWHLEGSGGNDAPGQYGAYFHDDGFLAFPGHVFSRSLPEVPETIELEVRTSTASGLLLWQGVEVGEAGQGKDFISLGLQDGHLVFRYQLGSGEARLVSEDPINDGEWHRVTALREGRRGSIQVDGEELVSGRSPGPNVAVNAKGSVYIGGAPDVATLTGGRFSSGITGCVKNLVLHSARPGAPPPQPLDLQHRAQAGANTRPCPS

SEQ ID NO: 3 - protein, 4383 aa, Mus musculus
MGQRAVGSLLLGLLLHARLLAVTHGLRAYDGLSLPEDTETVTASRYGWTYSYLSDDEDLLADDASGDGLGSGDVGSGDFQMVYFRALVNFTRSIEYSPQLEDASAKEFREVSEAVVEKLEPEYRKIPGDQIVSVVFIKELDGWVFVELDVGSEGNADGSQIQEVLHTVVSSGSIGPYVTSPWGFKFRRLGTVPQFPRVCTETEFACHSYNECVALEYRCDRRPDCRDMSDELNCEEPVPELSSSTPAVGKVSPLPLWPEAATTPPPPVTHGPQFLLPSVPGPSACGPQEASCHSGHCIPRDYLCDGQEDCRDGSDELGCASPPPCEPNEFACENGHCALKLWRCDGDFDCEDRTDEANCSVKQPGEVCGPTHFQCVSTNRCIPASFHCDEESDCPDRSDEFGCMPPQVVTPPQQSIQASRGQTVTFTCVATGVPTPIINWRLNWGHIPAHPRVTMTSEGGRGTLIIRDVKEADQGAYTCEAMNSRGMVFGIPDGVLELVPQRGPCPDGHFYLEDSASCLPCFCFGVTNVCQSSLRFRDQIRLSFDQPNDFKGVNVTMPSQPGVPPLSSTQLQIDPALQEFQLVDLSRRFLVHDAFWALPKQFLGNKVDSYGGFLRYKVRYELARGMLEPVQKPDVILVGAGYRLHSRGHTPTHPGTLNQRQVQLSEEHWVHESGRPVQRAEMLQALASLEAVLLQTVYNTKMASVGLSDIVMDTTVTHTTIHGRAHSVEECRCPIGYSGLSCESCDAHFTRVPGGPYLGTCSGCNCNGHASSCDPVYGHCLNCQHNTEGPQCDKCKPGFFGDATKATATACRPCPCPYIDASRRFSDTCFLDTDGQATCDACAPGYTGRRCESCAPGYEGNPIQPGGKCRPTTQEIVRCDERGSLGTSGETCRCKNNVVGRLCNECSDGSFHLSKQNPDGCLKCFCMGVSRQCSSSSWSRAQVLGASEQPSQFSLSNAAGTHTTSEGVSSPAPGELSFSSFHNLLSEPYFWSLPASFRGDKVTSYGGELRFTVTQRPRPSSAPLHRQPLVVLQGNNIVLEHHASRDPSPGQPSNFIVPFQEQAWQRPDGQPATREHLLMALAGIDALLIQASYTQQPAESRVSGISMDVAVPENTGQDSAREVEQCTCPPGYRGPSCQDCDTGYTRVPSGLYLGTCERCNCHGHSETCEPETGACQSCQHHTEGASCEQCQPGYYGDAQRGTPQDCQPCPCYGAPAAGQAAHTCFLDTDGHPTCDSCSPGHSGRHCERCAPGYYGNPSQGQPCHRDGQVPEVLGCGCDPHGSISSQCDAAGQCQCKAQVEGRTCSHCRPHHFHLSASNPEGCLPCFCMGVTQQCASSSYSRQLISTHFAPGDFQGFALVNPQRNSQLTGGFTVEPVHDGARLSFSNFAHLGQESFYWQLPEIYQGDKVAAYGGKLRYTLSYTAGPQGSPLLDPDIQITGNNIMLVASQPALQGPERRSYEIIFREEFWRRPDGQPATREHLLMALADLDELLVRATFSSVPRAASISAVSLEVAQPGPSSGPRALEVEECRCPPGYVGLSCQDCAPGYTRTGSGLYLGQCELCECNGHSDLCHPETGACSRCQHNTAGEFCELCATGYYGDATAGTPEDCQPCACPLTNPENMFSRTCESLGAGGYRCTACEPGYTGQYCEQCAPGYEGDPNVQGGRCQPLTKESLEVQIHPSRSVVPQGGPHSLRCQVSGSPPHYFYWSREDGRPLPSSAQQRHQGSELHFPSVQPSDAGVYICTCRNLIHTSNSRAELLVAEAPSKPITVTVEEQRSQSVRPGADVTFICTAKSKSPAYTLVWTRLHNGKLPSRAMDFNGILTIRNVQPSDAGTYVCTGSNMFAMDQGTATLHVQVSGTSTAPVASIHPPQLTVQPGQQAEFRCSATGNPTPMLEWIGGPSGQLPAKAQIHNGILRLPAIEPSDQGQYLCRALSSAGQHVARAMLQVHGGSGPRVQVSPERTQVHEGRTVRLYCRAAGVPSASITWRKEGGSLPPQARSENTDIPTLLIPAITAADAGFYLCVATSPTGTAQARIQVVVLSASGANSVPVRIESSSPSVTEGQTLDLNCAVMGLTYTQVTWYKRGGSLPPHAQVHGSRLRLPQVSPADSGDYVCRVESDVGPKEASIVVSVLHSPHSGPSYTPATSITPPIRIESSSSHVAEGQTLDLNCVVPGQAQVTWRKRGGSLPARHQTHGSLLRLHQVSPADSGEYVCHVVLGSEHTETSVLVTIEPAESIPAPGPAPPVRIEASSSTVTEGHMLDLNCVVAGQAHAQVTWYKRGGSLPARHQVRGSRLYILQASPADAGEYVCRAGNGQEATITVTVTRNHGANLAYPPGSTSPIRIESSSSHVAEGQTLDLNCVVQGQAHAQVTWHKRGGSLPARHQTHGSLLRLHQVSPVDSGEYVCRVEGGAVPLESSVLVTIEPAGTAPGVIPPVRIESSSSHVSEGQSLDLNCLVSGQTHPQISWHKRGGSLPARHQVHGSRLRLLQVTPTDSGEYVCRVVSGSGTQEASILVTIQQTLSPSHSQSVVHPVRIESSSPSLANGHTLDLNCLVASLTPHTITWYKRGGSLPSRHQIVGSRLRIPQVTPADSGEYVCHVSNGAGSQETSLIVTIESRGPSHVPSVSPPMRIETSSPTVTEGQTLDLNCVVVGRPQATITWYKRGGSLPFRHQAHGSRLRLHHMSVADSGEYVCRANNNIDAQETSIMISVSPSTNSPPAPASPAPIRIESSSSRVAEGQTLDLNCVVPGHAHAQVTWHKRGGSLPTHHQTHGSRLRLYQVSSADSGEYVCSVLSSSGPLEASVLVSITPAAANVHIPGEVPFPPIRIETSSSRVAEGQTLDLSCVVPGQAHAQVTWHKRGGSLPAGHQVHGHMLRLNRVSPADSGEYSCQVTGSSGTLEASVLVTIEASEPSPIPAPGLAQPVYIESSSSHLTEGQTVDLKCVVPGQAHAQVTWHKRGSSLPARHQTHGSLLRLYQLSPADSGEYVCQVAGSSHPEHEASFKLTVPSSQNSSFRLRSPVISIEPPSSTVQQGQDASFKCLIHEGATPIKVEWKIRDQELEDNVHISPNGSIITIVGTRPSNHGAYRCVASNVYGMAQSVVNLSVHGPPTVSVLPEGPVHVKMGKDITLECISSGEPRSSPRWTRLGIPVKLEPRMFGLMNSHAMLKIASVKPSDAGTYVCQAQNALGTAQKQVELIVDTGTVAPGAPQVQVEESELTLEAGHTATLHCSATGNPPPTIHWSKLRAPLPWQHRIEGNTLVIPRVAQQDSGQYICNATNSAGHTEATVVLHVESPPYATIIPEHTSAQPGNLVQLQCLAHGTPPLTYQWSLVGGVLPEKAVARNQVLRLEPTVPEDSGRYRCQVSNRVGSAEAFAQVLVQGSSSNLPDTSIPGGSTPTVQVTPQLETRNIGASVEFHCAVPNERGTHLRWLKEGGQLPPGHSVQDGVLRIQNLDQSCQGTYVCQAHGPWGQAQATAQLIVQALPSVLINVRTSVHSVVVGHSVEFECLALGDPKPQVTWSKVGGHLRPGIVQSGSIIRIAHVELADAGQYRCAATNAAGTTQSHVLLLVQALPQISTPPEIRVPAGSAAVFPCMASGYPTPAITWSKVDGDLPPDSRLENNMLMLPSVRPEDAGTYVCTATNRQGKVKAFAYLQVPERVIPYFTQTPYSFLPLPTIKDAYRKFEIKITFRPDSADGMLLYNGQKRSPTNLANRQPDFISFGLVGGRPEFRFDAGSGMATIRHPTPLALGQFHTVTLLRSLTQGSLIVGNLAPVNGTSQGKFQGLDLNEELYLGGYPDYGAIPKAGLSSGFVGCVRELRIQGEEVVFHDVNLTTHGISHCPTCQDRPCQNGGQCQDSESSSYTCVCPAGFTGSRCEHSQALHCHPEACGPDATCVNRPDGRGYTCRCHLGRSGVRCEEGVTVTTPSMSGAGSYLALPALTNMHHELRLDVEFKPLEPNGILLFSGGKSGPVEDFVSLAMVGGHLEFRYELGSGLAVLRSHEPLTLGRWHRVSAERLNKDGSLRVDGGRPVLRSSPGKSQGLNLHTLLYLGGVEPSVQLSPATNMSAHFHGCVGEVSVNGKRLDLTYSFLGSQGVGQCYDSSPCERQPCQNGATCMPAGEYEFQCLCQDGFKGDLCEHEENPCQLHEPCLNGGTCRGARCLCLPGFSGPRCQQGAGYGVVESDWHPEGSGGNDAPGQYGAYFYDNGFLGLPGNSFSRSLPEVPETIEFEVRTSTADGLLLWQGVVREASRSKDFISLGLQDGHLVFSYQLGSGEARLVSEDPINDGEWHRITALREGQRGSIQVDGEDLVTGRSPGPNVAVNTKDIIYIGGAPDVATLTRGKFSSGITGCIKNLVLHTARPGAPPPQPLDLQHRAQAGANTRPCPS

SEQ ID NO: 4 - protein, 2068 aa, Homo sapiens
MAGRSHPGPLRPLLPLLVVAACVLPGAGGTCPERALERREEEANVVLTGTVEEILNVDPVQHTYSCKVRVWRYLKGKDLVARESLLDGGNKVVISGFGDPLICDNQVSTGDTRIFFVNPAPPYLWPAHKNELMLNSSLMRITLRNLEEVEFCVEDKPGTHFTPVPPTPPDACRGMLCGFGAVCEPNAEGPGRASCVCKKSPCPSVVAPVCGSDASTYSNECELQRAQCSQQRRIRLLSRGPCGSRDPCSNVTCSFGSTCARSADGLTASCLCPATCRGAPEGTVCGSDGADYPGECQLLRRACARQENVFKKFDGPCDPCQGALPDPSRSCRVNPRTRRPEMLLRPESCPARQAPVCGDDGVTYENDCVMGRSGAARGLLLQKVRSGQCQGRDQCPEPCRFNAVCLSRRGRPRCSCDRVTCDGAYRPVCAQDGRTYDSDCWRQQAECRQQRAIPSKHQGPCDQAPSPCLGVQCAFGATCAVKNGQAACECLQACSSLYDPVCGSDGVTYGSACELEATACTLGREIQVARKGPCDRCGQCRFGALCEAETGRCVCPSECVALAQPVCGSDGHTYPSECMLHVHACTHQISLHVASAGPCETCGDAVCAFGAVCSAGQCVCPRCEHPPPGPVCGSDGVTYGSACELREAACLQQTQIEEARAGPCEQAECGSGGSGSGEDGDCEQELCRQRGGIWDEDSEDGPCVCDFSCQSVPGSPVCGSDGVTYSTECELKKARCESQRGLYVAAQGACRGPTFAPLPPVAPLHCAQTPYGCCQDNITAARGVGLAGCPSACQCNPHGSYGGTCDPATGQCSCRPGVGGLRCDRCEPGFWNFRGIVTDGRSGCTPCSCDPQGAVRDDCEQMTGLCSCKPGVAGPKCGQCPDGRALGPAGCEADASAPATCAEMRCEFGARCVEESGSAHCVCPMLTCPEANATKVCGSDGVTYGNECQLKTIACRQGLQISIQSLGPCQEAVAPSTHPTSASVTVTTPGLLLSQALPAPPGALPLAPSSTAHSQTTPPPSSRPRTTASVPRTTVWPVLTVPPTAPSPAPSLVASAFGESGSTDGSSDEELSGDQEASGGGSGGLEPLEGSSVATPGPPVERASCYNSALGCCSDGKTPSLDAEGSNCPATKVFQGVLELEGVEGQELFYTPEMADPKSELFGETARSIESTLDDLFRNSDVKKDFRSVRLRDLGPGKSVRAIVDVHFDPTTAFRAPDVARALLRQIQVSRRRSLGVRRPLQEHVRFMDFDWFPAFITGATSGAIAAGATARATTASRLPSSAVTPRAPHPSHTSQPVAKTTAAPTTRRPPTTAPSRVPGRRPPAPQQPPKPCDSQPCFHGGTCQDWALGGGFTCSCPAGRGGAVCEKVLGAPVPAFEGRSFLAFPTLRAYHTLRLALEFRALEPQGLLLYNGNARGKDFLALALLDGRVQLRFDTGSGPAVLTSAVPVEPGQWHRLELSRHWRRGTLSVDGETPVLGESPSGTDGLNLDTDLFVGGVPEDQAAVALERTFVGAGLRGCIRLLDVNNQRLELGIGPGAATRGSGVGECGDHPCLPNPCHGGAPCQNLEAGRFHCQCPPGRVGPTCADEKSPCQPNPCHGAAPCRVLPEGGAQCECPLGREGTFCQTASGQDGSGPFLADFNGFSHLELRGLHTFARDLGEKMALEVVFLARGPSGLLLYNGQKTDGKGDFVSLALRDRRLEFRYDLGKGAAVIRSREPVTLGAWTRVSLERNGRKGALRVGDGPRVLGESPKSRKVPHTVLNLKEPLYVGGAPDFSKLARAAAVSSGFDGAIQLVSLGGRQLLTPEHVLRQVDVTSFAGHPCTRASGHPCLNGASCVPREAAYVCLCPGGFSGPHCEKGLVEKSAGDVDTLAFDGRTFVEYLNAVTESELANEIPVPETLDSGALHSEKALQSNHFELSLRTEATQGLVLWSGKATERADYVALAIVDGHLQLSYNLGSQPVVLRSTVPVNTNRWLRVVAHREQREGSLQVGNEAPVTGSSPLGATQLDTDGALWLGGLPELPVGPALPKAYGTGFVGCLRDVVVGRHPLHLLEDAVTKPELRPCPTP

SEQ ID NO: 5 - protein, 2045 aa, Homo sapiens
MAGRSHPGPLRPLLPLLVVAACVLPGAGGTCPERALERREEEANVVLTGTVEEILNVDPVQHTYSCKVRVWRYLKGKDLVARESLLDGGNKVVISGFGDPLICDNQVSTGDTRIFFVNPAPPYLWPAHKNELMLNSSLMRITLRNLEEVEFCVEDKPGTHFTPVPPTPPDACRGMLCGFGAVCEPNAEGPGRASCVCKKSPCPSVVAPVCGSDASTYSNECELQRAQCSQQRRIRLLSRGPCGSRDPCSNVTCSFGSTCARSADGLTASCLCPATCRGAPEGTVCGSDGADYPGECQLLRRACARQENVFKKFDGPCDPCQGALPDPSRSCRVNPRTRRPEMLLRPESCPARQAPVCGDDGVTYENDCVMGRSGAARGLLLQKVRSGQCQGRDQCPEPCRFNAVCLSRRGRPRCSCDRVTCDGAYRPVCAQDGRTYDSDCWRQQAECRQQRAIPSKHQGPCDQAPSPCLGVQCAFGATCAVKNGQAACECLQACSSLYDPVCGSDGVTYGSACELEATACTLGREIQVARKGPCDRCGQCRFGALCEAETGRCVCPSECVALAQPVCGSDGHTYPSECMLHVHACTHQISLHVASAGPCETCGDAVCAFGAVCSAGQCVCPRCEHPPPGPVCGSDGVTYGSACELREAACLQQTQIEEARAGPCEQAECGSGGSGSGEDGDCEQELCRQRGGIWDEDSEDGPCVCDFSCQSVPGSPVCGSDGVTYSTECELKKARCESQRGLYVAAQGACRGPTFAPLPPVAPLHCAQTPYGCCQDNITAARGVGLAGCPSACQCNPHGSYGGTCDPATGQCSCRPGVGGLRCDRCEPGFWNFRGIVTDGRSGCTPCSCDPQGAVRDDCEQMTGLCSCKPGVAGPKCGQCPDGRALGPAGCEADASAPATCAEMRCEFGARCVEESGSAHCVCPMLTCPEANATKVCGSDGVTYGNECQLKTIACRQGLQISIQSLGPCQEAVAPSTHPTSASVTVTTPGLLLSQALPAPPGALPLAPSSTAHSQTTPPPSSRPRTTASVPRTTVWPVLTVPPTAPSPAPSLVASAFGESGSTDGSSDEELSGDQEASGGGSGGLEPLEGSSVATPGPPVERASCYNSALGCCSDGKTPSLDAEGSNCPATKVFQGVLELEGVEGQELFYTPEMADPKSELFGETARSIESTLDDLFRNSDVKKDFRSVRLRDLGPGKSVRAIVDVHFDPTTAFRAPDVARALLRQIQVSRRRSLGVRRPLQEHVRFMDFDWFPAFITGATSGAIAAGATARATTASRLPSSAVTPRAPHPSHTSQPVAKTTAAPTTRRPPTTAPSRVPGRRPPAPQQPPKPCDSQPCFHGGTCQDWALGGGFTCSCPAGRGGAVCEKVLGAPVPAFEGRSFLAFPTLRAYHTLRLALEFRALEPQGLLLYNGNARGKDFLALALLDGRVQLRFDTGSGPAVLTSAVPVEPGQWHRLELSRHWRRGTLSVDGETPVLGESPSGTDGLNLDTDLFVGGVPEDQAAVALERTFVGAGLRGCIRLLDVNNQRLELGIGPGAATRGSGVGECGDHPCLPNPCHGGAPCQNLEAGRFHCQCPPGRVGPTCADEKSPCQPNPCHGAAPCRVLPEGGAQCECPLGREGTFCQTASGQDGSGPFLADFNGFSHLELRGLHTFARDLGEKMALEVVFLARGPSGLLLYNGQKTDGKGDFVSLALRDRRLEFRYDLGKGAAVIRSREPVTLGAWTRVSLERNGRKGALRVGDGPRVLGESPVPHTVLNLKEPLYVGGAPDFSKLARAAAVSSGFDGAIQLVSLGGRQLLTPEHVLRQVDVTSFAGHPCTRASGHPCLNGASCVPREAAYVCLCPGGFSGPHCEKGLVEKSAGDVDTLAFDGRTFVEYLNAVTESEKALQSNHFELSLRTEATQGLVLWSGKATERADYVALAIVDGHLQLSYNLGSQPVVLRSTVPVNTNRWLRVVAHREQREGSLQVGNEAPVTGSSPLGATQLDTDGALWLGGLPELPVGPALPKAYGTGFVGCLRDVVVGRHPLHLLEDAVTKPELRPCPTP

SEQ ID NO: 6 - protein, 1940 aa, Rattus norvegicus
MPPLPLEHRPRQEPGASMLVRYFMIPCNICLILLATSTLGFAVLLFLSNYKPGIHFTPAPPTPPDVCRGMLCGFGAVCEPSVEDPGRASCVCKKNACPATVAPVCGSDASTYSNECELQRAQCNQQRRIRLLRQGPCGSRDPCANVTCSFGSTCVPSADGQTASCLCPTTCFGAPDGTVCGSDGVDYPSECQLLSHACASQEHIFKKFNGPCDPCQGSMSDLNHICRVNPRTRHPEMLLRPENCPAQHTPICGDDGVTYENDCVMSRIGATRGLLLQKVRSGQCQTRDQCPETCQFNSVCLSRRGRPHCSCDRVTCDGSYRPVCAQDGHTYNNDCWRQQAECRQQRAIPPKHQGPCDQTPSPCHGVQCAFGAVCTVKNGKAECECQRVCSGIYDPVCGSDGVTYGSVCELESMACTLGREIQVARRGPCDPCGQCRFGSLCEVETGRCVCPSECVESAQPVCGSDGHTYASECELHVHACTHQISLYVASAGHCQTCGEKVCTFGAVCSAGQCVCPRCEHPPPGPVCGSDGVTYLSACELREAACQQQVQIEEAHAGPCEPAECGSGGSGSGEDDECEQELCRQRGGIWDEDSEDGPCVCDFSCQSVPRSPVCGSDGVTYGTECDLKKARCESQQELYVAAQGACRGPTLAPLLPVAFPHCAQTPYGCCQDNFTAAQGVGLAGCPSTCHCNPHGSYSGTCDPATGQCSCRPGVGGLRCDRCEPGFWNFRGIVTDGHSGCTPCSCDPRGAVRDDCEQMTGLCSCRPGVAGPKCGQCPDGQVLGHLGCEADPMTPVTCVEIHCEFGASCVEKAGFAQCICPTLTCPEANSTKVCGSDGVTYGNECQLKAIACRQRLDISTQSLGPCQESVTPGASPTSASMTTPRHILSKTLPFPHNSLPLSPGSTTHDWPTPLPISPHTTVSIPRSTAWPVLTVPPTAAASDVTSLATSIFSESGSANGSGDEELSGDEEASGGGSGGLEPPVGSIVVTHGPPIERASCYNSPLGCCSDGKTPSLDSEGSNCPATKAFQGVLELEGVEGQELFYTPEMADPKSELFGETARSIESTLDDLFRNSDVKKDFWSVRLRELGPGKLVRAIVDVHFDPTTAFQASDVGQALLRQIQVSRPWALAVRRPLQEHVRFLDFDWFPTFFTGAATGTTAAMATARATTVSRLPASSVTPRVYPSHTSRPVGRTTAPPTTRRPPTTATNMDRPRTPGHQQPSKSCDSQPCLHGGTCQDQDSGKGFTCSCTAGRGGSVCEKVQPPSMPAFKGHSFLAFPTLRAYHTLRLALEFRALETEGLLLYNGNARGKDFLALALLDGRVQFRFDTGSGPAVLTSLVPVEPGRWHRLELSRHWRQGTLSVDGETPVVGESPSGTDGLNLDTNLYVGGIPEEQVAMVLDRTSVGVGLKGCIRMLDINNQQLELSDWQRAAVQSSGVGECGDHPCLPNPCHGGALCQALEAGMFLCQCPPGRFGPTCADEKSPCQPNPCHGAAPCRVLSSGGAKCECPLGRSGTFCQTVLETAGSRPFLADFNGFSYLELKGLHTFERDLGEKMALEMVFLARGPSGLLLYNGQKTDGKGDFVSLALHNRHLEFCYDLGKGAAVIRSKEPIALGTWVRVFLERNGRKGALQVGDGPRVLGESPKSRKVPHTMLNLKEPLYIGGAPDFSKLARGAAVSSGFSGVIQLVSLRGHQLLTQEHVLRAVDVSPFADHPCTQALGNPCLNGGSCVPREATYECLCPGGFSGLHCEKGLVEKSVGDLETLAFDGRTYIEYLNAVIESEKALQSNHFELSLRTEATQGLVLWIGKAAERADYMALAIVDGHLQLSYDLGSQPVVLRSTVKVNTNRWLRIRAHREHREGSLQVGNEAPVTGSSPLGATQLDTDGALWLGGLQKLPVGQALPKAYGTGFVGCLRDVVVGHRQLHLLEDAVTKPELRPCPTP

SEQ ID NO: 7 - protein, 452 aa, Artificial Sequence
  Amino acid sequence encoding Minimal human Agrin human sequence - G1 + G2
GAPVPAFEGRSFLAFPTLRAYHTLRLALEFRALEPQGLLLYNGNARGKDFLALALLDGRVQLRFDTGSGPAVLTSAVPVEPGQWHRLELSRHWRRGTLSVDGETPVLGESPSGTDGLNLDTDLFVGGVPEDQAAVALERTFVGAGLRGCIRLLDVNNQRLELGIGPGAATRGSGVGECGDHPCLPNPCHGGAPCQNLEAGRFHCQCPPGRVGPTCADEKSPCQPNPCHGAAPCRVLPEGGAQCECPLGREGTFCQTASGQDGSGPFLADFNGFSHLELRGLHTFARDLGEKMALEVVFLARGPSGLLLYNGQKTDGKGDFVSLALRDRRLEFRYDLGKGAAVIRSREPVTLGAWTRVSLERNGRKGALRVGDGPRVLGESPKSRKVPHTVLNLKEPLYVGGAPDFSKLARAAAVSSGFDGAIQLVSLGGRQLLTPEHVLRQVDVTSFAGHPC

SEQ ID NO: 8 - protein, 188 aa, Artificial Sequence
  Amino acid sequence encoding Minimal human Agrin human sequence -  G2 only
PFLADFNGFSHLELRGLHTFARDLGEKMALEVVFLARGPSGLLLYNGQKTDGKGDFVSLALRDRRLEFRYDLGKGAAVIRSREPVTLGAWTRVSLERNGRKGALRVGDGPRVLGESPKSRKVPHTVLNLKEPLYVGGAPDFSKLARAAAVSSGFDGAIQLVSLGGRQLLTPEHVLRQVDVTSFAGHPC

SEQ ID NO: 9 - protein, 2034 aa, Mus musculus
MVRPRLSFPAPLLPLLLLLAAAAPAVPGASGTCPERALERREEEANVVLTGTVEEILNVDPVQHTYSCKVRVWRYLKGKDVVAQESLLDGGNKVVIGGFGDPLICDNQVSTGDTRIFFVNPAPPYLWPAHKNELMLNSSLMRITLRNLEEVEFCVEDKPGIHFTAAPSMPPDVCRGMLCGFGAVCEPSVEDPGRASCVCKKNVCPAMVAPVCGSDASTYSNECELQRAQCNQQRRIRLLRQGPCGSRDPCANVTCSFGSTCVPSADGQTASCLCPTTCFGAPDGTVCGSDGVDYPSECQLLRHACANQEHIFKKFDGPCDPCQGSMSDLNHICRVNPRTRHPEMLLRPENCPAQHTPICGDDGVTYENDCVMSRIGAARGLLLQKVRSGQCQTRDQCPETCQFNSVCLSRRGRPHCSCDRVTCDGAYRPVCAQDGHTYDNDCWRQQAECRQQQTIPPKHQGPCDQTPSPCRGAQCAFGATCTVKNGKAVCECQRVCSGGYDPVCGSDGVTYGSVCELESMACTLGREIRVARRGPCDRCGQCRFGSLCEVETGRCVCPSECVESAQPVCGSDGHTYASECELHVHACTHQISLYVASAGHCQTCGETVCTFGAVCSAGQCVCPRCEHPPPGPVCGSDGVTYLSACELREAACQQQVQIEEARAGPCEPAECGSGGSGSGEDNACEQELCRQHGGVWDEDSEDGPCVCDFSCQSVLKSPVCGSDGVTYSTECHLKKARCEARQELYVAAQGACRGPTLAPLLPMASPHCAQTPYGCCQDNVTAAQGVGLAGCPSTCHCNPHGSYSGTCDPVTGQCSCRPGVGGLRCDRCEPGFWNFRGIVTDGHSGCTPCSCDPRGAVRDDCEQMTGLCSCRPGVAGPKCGQCPDGQALGHLGCEADPTTPVTCVEMHCEFGASCVEEAGFAQCVCPTLTCPEANSTKVCGSDGVTYGNECQLKTIACRQRLDISIQSLGPCRESVAPGVSPTSASMTTPRHILSRTLASPHSSLPLSPSTTAHDWPTPLPTSPQTVVGTPRSTAATPSDVASLATAIFRESGSTNGSGDEELSGDEEASGGGSGGLEPPVGSVVVTHGPPIERASCYNSPLGCCSDGKTPSLDSEGSNCPATKAFQGVLELEGVEGQELFYTPEMADPKSELFGETARSIESTLDDLFRNSDVKKDFWSIRLRELGPGKLVRAIVDVHFDPTTAFQAPDVGQALLQQIQVSRPWALAVRRPLREHVRFLDFDWFPTFFTGAATGTTAAVATARATTVSRLSASSVTPRVYPSYTSRPVGRTTAPLTTRRPPTTTASIDRPRTPGPQRPPKSCDSQPCLHGGTCQDLDSGKGFSCSCTAGRAGTVCEKVQLPSVPAFKGHSFLAFPTLRAYHTLRLALEFRALETEGLLLYNGNARGKDFLALALLDGHVQFRFDTGSGPAVLTSLVPVEPGRWHRLELSRHWRQGTLSVDGEAPVVGESPSGTDGLNLDTKLYVGGLPEEQVATVLDRTSVGIGLKGCIRMLDINNQQLELSDWQRAVVQSSGVGECGDHPCSPNPCHGGALCQALEAGVFLCQCPPGRFGPTCADEKNPCQPNPCHGSAPCHVLSRGGAKCACPLGRSGSFCETVLENAGSRPFLADFNGFSYLELKGLHTFERDLGEKMALEMVFLARGPSGLLLYNGQKTDGKGDFVSLALHNRHLEFRYDLGKGAAIIRSKEPIALGTWVRVFLERNGRKGALQVGDGPRVLGESPVPHTMLNLKEPLYVGGAPDFSKLARGAAVASGFDGAIQLVSLRGHQLLTQEHVLRAVDVAPFAGHPCTQAVDNPCLNGGSCIPREATYECLCPGGFSGLHCEKGIVEKSVGDLETLAFDGRTYIEYLNAVTESEKALQSNHFELSLRTEATQGLVLWIGKVGERADYMALAIVDGHLQLSYDLGSQPVVLRSTVKVNTNRWLRVRAHREHREGSLQVGNEAPVTGSSPLGATQLDTDGALWLGGLQKLPVGQALPKAYGTGFVGCLRDVVVGHRQLHLLEDAVTKPELRPCPTL

SEQ ID NO: 10 - DNA, 20 bp, Artificial Sequence
  Single strand DNA oligonucleotide
gtcggctcgcggcaaaaagc

SEQ ID NO: 11 - DNA, 20 bp, Artificial Sequence
  Single strand DNA oligonucleotide
gacatcaaagagaagctgtg

SEQ ID NO: 12 - DNA, 21 bp, Artificial Sequence
  Single strand DNA oligonucleotide
actccataccgataaaggaag

SEQ ID NO: 13 - DNA, 20 bp, Artificial Sequence
  Single strand DNA oligonucleotide
gagagaaagaaaccagagtg

SEQ ID NO: 14 - DNA, 21 bp, Artificial Sequence
  Single strand DNA oligonucleotide
gtctagcaggttcttgaaatc

SEQ ID NO: 15 - DNA, 20 bp, Artificial Sequence
  Single strand DNA oligonucleotide
aattcaagatgcagaagctg

SEQ ID NO: 16 - DNA, 20 bp, Artificial Sequence
  Single strand DNA oligonucleotide
gaattttgaggtctctgctg

SEQ ID NO: 17 - DNA, 21 bp, Artificial Sequence
  Single strand DNA oligonucleotide
ccagaaacatcatcataaccg

SEQ ID NO: 18 - DNA, 20 bp, Artificial Sequence
  Single strand DNA oligonucleotide
catcgccaccttaatagttg

SEQ ID NO: 19 - DNA, 20 bp, Artificial Sequence
  Single strand DNA oligonucleotide
gtcaggctggtcaccttctg

SEQ ID NO: 20 - DNA, 20 bp, Artificial Sequence
  Single strand DNA oligonucleotide
aactcttggcaccatgaacc

SEQ ID NO: 21 - DNA, 22 bp, Artificial Sequence
  Single strand DNA oligonucleotide
gctgggctccctggacattgac

SEQ ID NO: 22 - DNA, 22 bp, Artificial Sequence
  Single strand DNA oligonucleotide
cctgggcctggattctggtgat

SEQ ID NO: 23 - DNA, 25 bp, Artificial Sequence
  Single strand DNA oligonucleotide
ccagtgtgaacttgattttgatgaa

SEQ ID NO: 24 - DNA, 27 bp, Artificial Sequence
  Single strand DNA oligonucleotide
aacataacttgggagacagagacatct

SEQ ID NO: 25 - DNA, 20 bp, Artificial Sequence
  Single strand DNA oligonucleotide
catcgacgcccagatgaaga

SEQ ID NO: 26 - DNA, 20 bp, Artificial Sequence
  Single strand DNA oligonucleotide
tggtgaacagggtcccaaac

SEQ ID NO: 27 - DNA, 18 bp, Artificial Sequence
  Single strand DNA oligonucleotide
cggaggagcgaacacctg

SEQ ID NO: 28 - DNA, 20 bp, Artificial Sequence
  Single strand DNA oligonucleotide
gttggatcctcaccctctgc

SEQ ID NO: 29 - DNA, 20 bp, Artificial Sequence
  Single strand DNA oligonucleotide
ttcgatggtccttgtgaccc

SEQ ID NO: 30 - DNA, 20 bp, Artificial Sequence
  Single strand DNA oligonucleotide
agataggtgtgtgttgggcg

SEQ ID NO: 31 - DNA, 20 bp, Artificial Sequence
  Single strand DNA oligonucleotide
gtgcccatcgtcaacctgaa

SEQ ID NO: 32 - DNA, 20 bp, Artificial Sequence
  Single strand DNA oligonucleotide
agttgaccctgggagccaga

SEQ ID NO: 33 - DNA, 22 bp, Artificial Sequence
  Single strand DNA oligonucleotide
ccttctggcacaagtctcttgg

SEQ ID NO: 34 - DNA, 22 bp, Artificial Sequence
  Single strand DNA oligonucleotide
tcgaagatgacactggcatcgg

SEQ ID NO: 35 - DNA, 20 bp, Artificial Sequence
  Single strand DNA oligonucleotide
tcgagcttgatctctcctat

SEQ ID NO: 36 - DNA, 20 bp, Artificial Sequence
  Single strand DNA oligonucleotide
tggtcccaggtcttacagaa

SEQ ID NO: 37 - DNA, 20 bp, Artificial Sequence
  Single strand DNA oligonucleotide
tggccggcagcgtttctgag

SEQ ID NO: 38 - protein, 4380 aa, Homo sapiens
MGWRAAGALLLALLLHGRLLAVTHGLRAYDGLSLPEDIETVTASQMRWTHSYLSDDEDMLADSISGDDLGSGDLGSGDFQMVYFRALVNFTRSIEYSPQLEDAGSREFREVSEAVVDTLESEYLKIPGDQVVSVVFIKELDGWVFVELDVGSEGNADGAQIQEMLLRVISSGSVASYVTSPQGFQFRRLGTVPQFPRACTEAEFACHSYNECVALEYRCDRRPDCRDMSDELNCEEPVLGISPTFSLLVETTSLPPRPETTIMRQPPVTHAPQPLLPGSVRPLPCGPQEAACRNGHCIPRDYLCDGQEDCEDGSDELDCGPPPPCEPNEFPCGNGHCALKLWRCDGDFDCEDRTDEANCPTKRPEEVCGPTQFRCVSTNMCIPASFHCDEESDCPDRSDEFGCMPPQVVTPPRESIQASRGQTVTFTCVAIGVPTPIINWRLNWGHIPSHPRVTVTSEGGRGTLIIRDVKESDQGAYTCEAMNARGMVFGIPDGVLELVPQRGPCPDGHFYLEHSAACLPCFCFGITSVCQSTRRFRDQIRLRFDQPDDFKGVNVTMPAQPGTPPLSSTQLQIDPSLHEFQLVDLSRRFLVHDSFWALPEQFLGNKVDSYGGSLRYNVRYELARGMLEPVQRPDVVLMGAGYRLLSRGHTPTQPGALNQRQVQFSEEHWVHESGRPVQRAELLQVLQSLEAVLIQTVYNTKMASVGLSDIAMDTTVTHATSHGRAHSVEECRCPIGYSGLSCESCDAHFTRVPGGPYLGTCSGCNCNGHASSCDPVYGHCLNCQHNTEGPQCNKCKAGFFGDAMKATATSCRPCPCPYIDASRRFSDTCFLDTDGQATCDACAPGYTGRRCESCAPGYEGNPIQPGGKCRPVNQEIVRCDERGSMGTSGEACRCKNNVVGRLCNECADGSFHLSTRNPDGCLKCFCMGVSRHCTSSSWSRAQLHGASEEPGHFSLTNAASTHTTNEGIFSPTPGELGFSSFHRLLSGPYFWSLPSRFLGDKVTSYGGELRFTVTQRSQPGSTPLHGQPLVVLQGNNIILEHHVAQEPSPGQPSTFIVPFREQAWQRPDGQPATREHLLMALAGIDTLLIRASYAQQPAESRVSGISMDVAVPEETGQDPALEVEQCSCPPGYRGPSCQDCDTGYTRTPSGLYLGTCERCSCHGHSEACEPETGACQGCQHHTEGPRCEQCQPGYYGDAQRGTPQDCQLCPCYGDPAAGQAAHTCFLDTDGHPTCDACSPGHSGRHCERCAPGYYGNPSQGQPCQRDSQVPGPIGCNCDPQGSVSSQCDAAGQCQCKAQVEGLTCSHCRPHHFHLSASNPDGCLPCFCMGITQQCASSAYTRHLISTHFAPGDFQGFALVNPQRNSRLTGEFTVEPVPEGAQLSFGNFAQLGHESFYWQLPETYQGDKVAAYGGKLRYTLSYTAGPQGSPLSDPDVQITGNNIMLVASQPALQGPERRSYEIMFREEFWRRPDGQPATREHLLMALADLDELLIRATFSSVPLAASISAVSLEVAQPGPSNRPRALEVEECRCPPGYIGLSCQDCAPGYTRTGSGLYLGHCELCECNGHSDLCHPETGACSQCQHNAAGEFCELCAPGYYGDATAGTPEDCQPCACPLTNPENMFSRTCESLGAGGYRCTACEPGYTGQYCEQCGPGYVGNPSVQGGQCLPETNQAPLVVEVHPARSIVPQGGSHSLRCQVSGSPPHYFYWSREDGRPVPSGTQQRHQGSELHFPSVQPSDAGVYICTCRNLHQSNTSRAELLVTEAPSKPITVTVEEQRSQSVRPGADVTFICTAKSKSPAYTLVWTRLHNGKLPTRAMDFNGILTIRNVQLSDAGTYVCTGSNMFAMDQGTATLHVQASGTLSAPVVSIHPPQLTVQPGQLAEFRCSATGSPTPTLEWTGGPGGQLPAKAQIHGGILRLPAVEPTDQAQYLCRAHSSAGQQVARAVLHVHGGGGPRVQVSPERTQVHAGRTVRLYCRAAGVPSATITWRKEGGSLPPQARSERTDIATLLIPAITTADAGFYLCVATSPAGTAQARIQVVVLSASDASPPPVKIESSSPSVTEGQTLDLNCVVAGSAHAQVTWYRRGGSLPPHTQVHGSRLRLPQVSPADSGEYVCRVENGSGPKEASITVSVLHGTHSGPSYTPVPGSTRPIRIEPSSSHVAEGQTLDLNCVVPGQAHAQVTWHKRGGSLPARHQTHGSLLRLHQVTPADSGEYVCHVVGTSGPLEASVLVTIEASVIPGPIPPVRIESSSSTVAEGQTLDLSCVVAGQAHAQVTWYKRGGSLPARHQVRGSRLYIFQASPADAGQYVCRASNGMEASITVTVTGTQGANLAYPAGSTQPIRIEPSSSQVAEGQTLDLNCVVPGQSHAQVTWHKRGGSLPVRHQTHGSLLRLYQASPADSGEYVCRVLGSSVPLEASVLVTIEPAGSVPALGVTPTVRIESSSSQVAEGQTLDLNCLVAGQAHAQVTWHKRGGSLPARHQVHGSRLRLLQVTPADSGEYVCRVVGSSGTQEASVLVTIQQRLSGSHSQGVAYPVRIESSSASLANGHTLDLNCLVASQAPHTITWYKRGGSLPSRHQIVGSRLRIPQVTPADSGEYVCHVSNGAGSRETSLIVTIQGSGSSHVPSVSPPIRIESSSPTVVEGQTLDLNCVVARQPQAIITWYKRGGSLPSRHQTHGSHLRLHQMSVADSGEYVCRANNNIDALEASIVISVSPSAGSPSAPGSSMPIRIESSSSHVAEGETLDLNCVVPGQAHAQVTWHKRGGSLPSHHQTRGSRLRLHHVSPADSGEYVCRVMGSSGPLEASVLVTIEASGSSAVHVPAPGGAPPIRIEPSSSRVAEGQTLDLKCVVPGQAHAQVTWHKRGGNLPARHQVHGPLLRLNQVSPADSGEYSCQVTGSSGTLEASVLVTIEPSSPGPIPAPGLAQPIYIEASSSHVTEGQTLDLNCVVPGQAHAQVTWYKRGGSLPARHQTHGSQLRLHLVSPADSGEYVCRAASGPGPEQEASFTVTVPPSEGSSYRLRSPVISIDPPSSTVQQGQDASFKCLIHDGAAPISLEWKTRNQELEDNVHISPNGSIITIVGTRPSNHGTYRCVASNAYGVAQSVVNLSVHGPPTVSVLPEGPVWVKVGKAVTLECVSAGEPRSSARWTRISSTPAKLEQRTYGLMDSHAVLQISSAKPSDAGTYVCLAQNALGTAQKQVEVIVDTGAMAPGAPQVQAEEAELTVEAGHTATLRCSATGSPAPTIHWSKLRSPLPWQHRLEGDTLIIPRVAQQDSGQYICNATSPAGHAEATIILHVESPPYATTVPEHASVQAGETVQLQCLAHGTPPLTFQWSRVGSSLPGRATARNELLHFERAAPEDSGRYRCRVTNKVGSAEAFAQLLVQGPPGSLPATSIPAGSTPTVQVTPQLETKSIGASVEFHCAVPSDRGTQLRWFKEGGQLPPGHSVQDGVLRIQNLDQSCQGTYICQAHGPWGKAQASAQLVIQALPSVLINIRTSVQTVVVGHAVEFECLALGDPKPQVTWSKVGGHLRPGIVQSGGVVRIAHVELADAGQYRCTATNAAGTTQSHVLLLVQALPQISMPQEVRVPAGSAAVFPCIASGYPTPDISWSKLDGSLPPDSRLENNMLMLPSVRPQDAGTYVCTATNRQGKVKAFAHLQVPERVVPYFTQTPYSFLPLPTIKDAYRKFEIKITFRPDSADGMLLYNGQKRVPGSPTNLANRQPDFISFGLVGGRPEFRFDAGSGMATIRHPTPLALGHFHTVTLLRSLTQGSLIVGDLAPVNGTSQGKFQGLDLNEELYLGGYPDYGAIPKAGLSSGFIGCVRELRIQGEEIVFHDLNLTAHGISHCPTCRDRPCQNGGQCHDSESSSYVCVCPAGFTGSRCEHSQALHCHPEACGPDATCVNRPDGRGYTCRCHLGRSGLRCEEGVTVTTPSLSGAGSYLALPALTNTHHELRLDVEFKPLAPDGVLLFSGGKSGPVEDFVSLAMVGGHLEFRYELGSGLAVLRSAEPLALGRWHRVSAERLNKDGSLRVNGGRPVLRSSPGKSQGLNLHTLLYLGGVEPSVPLSPATNMSAHFRGCVGEVSVNGKRLDLTYSFLGSQGIGQCYDSSPCERQPCQHGATCMPAGEYEFQCLCRDGFKGDLCEHEENPCQLREPCLHGGTCQGTRCLCLPGFSGPRCQQGSGHGIAESDWHLEGSGGNDAPGQYGAYFHDDGFLAFPGHVFSRSLPEVPETIELEVRTSTASGLLLWQGVEVGEAGQGKDFISLGLQDGHLVFRYQLGSGEARLVSEDPINDGEWHRVTALREGRRGSIQVDGEELVSGRSPGPNVAVNAKGSVYIGGAPDVATLTGGRFSSGITGCVKNLVLHSARPGAPPPQPLDLQHRA